(19) United States
(12) Patent Application Publication
(10) Pub. No.: US 2026/0337288 A1
(43) Pub. Date: Oct. 29, 2026

(54) DC-TARGETING VACCINE AGAINST NIPAH VIRUS INFECTION

(71) Applicants: INSTITUT NATIONAL DE LA SANTÉ ET DE LA RECHERCHE MÉDICALE, Paris (FR); ASSISTANCE PUBLIQUE-HÔPITAUX DE PARIS (APHP), Paris (FR); UNIVERSITÉ PARIS-EST CRÉTEIL VAL DE MARNE, Creteil (FR)

(72) Inventors: Yves LEVY, Creteil (FR); Mathieu SURENAUD, Creteil Cedex (FR); Sylvain CARDINAUD, Créteil (FR); Véronique GODOT, Creteil - Cedex (FR); Yadira PASTOR, Pamplona (ES)

(21) Appl. No.: 19/111,396

(22) PCT Filed: Oct. 4, 2023
(86) PCT No.: PCT/EP2023/077477

§ 371 Date: Mar. 13, 2025

(30) Foreign Application Priority Data
Oct. 5, 2022 (EP) 22306485.8

(51) Int. Cl. C07K 16/28; A61K 39/00; A61K 39/155; A61P 37/04; C07K 14/005

(52) U.S. Cl. CPC C07K 16/2878; A61K 39/155; A61P 37/04; C07K 14/005; A61K 2039/505; C07K 2319/00; C12N 2760/18222; C12N 2760/18234

(57) ABSTRACT

Nipah virus (NiV) is a recently emergent, highly pathogenic, zoonotic paramyxovirus. The inventors now designed an anti-CD40 mAb associated either with the Niv G ectodomain protein (Generation-1 vaccine), or the Niv G ectodomain protein and down-selected epitopes from the Niv F and N proteins (Generation-2 vaccine). Quality controls were performed on vaccine batches. The immunogenicity of both vaccines has been tested in hCD40Tg mice. A dose-dependent IFNg T cell response to the antigen was observed. Three weeks post-boost, specific IgG were detectable in groups immunized with 10 ug of vaccine. B cell responses were markedly improved 1 week post-boost. All samples at week −4 showed a neutralization with an average titer at 1:500. The inventors also demonstrated the potency of an innovative DC-targeting vaccine candidate to prevent NiV-B infection in challenge experiments in an AGM model. Responses were showed to cross-neutralize multiple strains of NiV, but also HeV. Targeting Nipah virus antigens to professional APCs can be efficiently used as a prophylactic means against a Nipah virus challenge at a lethal dose. Accordingly, the present invention relates to antibodies that are directed against a surface antigen of an antigen presenting cell wherein the heavy chain and/or the light chain is conjugated or fused to the Nipah virus antigenic polypeptides.

### FIELD OF THE INVENTION

The present invention is in the field of medicine, in particular virology.

### BACKGROUND OF THE INVENTION

Nipah virus (NiV) is a recently emergent, highly pathogenic, zoonotic paramyxovirus first recognized following a 1998-99 outbreak of severe febrile encephalitis in Malaysia and Singapore (Chua, K. B., et al. “Nipah virus: a recently emergent deadly paramyxovirus.” Science 288.5470 (2000): 1432-1435.). The Bangladesh strain (NiVB) has caused repeated outbreaks, varying in number, in Bangladesh and northeast India with outbreaks occurring almost every year between 2001-2015. The outbreaks of NiVB have had high case fatality rate averaging about 75% (Lo, Michael K., et al. “Characterization of Nipah virus from outbreaks in Bangladesh, 2008-2010.” Emerging infectious diseases 18.2 (2012): 248.) with human-to-human transmission often observed (Gurley, Emily S., et al. “Person-to-person transmission of Nipah virus in a Bangladeshi community.” Emerging infectious diseases 13.7 (2007): 1031.). Multiple candidate vaccines exist, but all are in the preclinical stages. Although rVSV vectors expressing Nipah virus G (or F) are prime candidates for new ‘emergency vaccines’ to be utilized for outbreak management (Foster, Stephanie L., et al. “A recombinant VSV-vectored vaccine rapidly protects nonhuman primates against lethal Nipah virus disease.” Proceedings of the National Academy of Sciences 119.12 (2022): e2200065119.), concerns remain about safety, development and inadequate transport/storage in affected areas. Thus, improving the ability of vaccines to induce strong, cellular and humoral immune responses, placing them as a new generic vaccine platform for prophylactic strategies but also at the heart of a therapeutic arsenal remains the challenge to rapidly and efficiently respond to Nipah.

### SUMMARY OF THE INVENTION

The present invention is defined by the claims. In particular, the present invention relates to antibodies that are directed against a surface antigen of an antigen presenting cell wherein the heavy chain and/or the light chain is conjugated or fused to the Nipah virus antigenic polypeptides.

### DETAILED DESCRIPTION OF THE INVENTION

### Definitions

As used herein, the term “subject” or “subject in need thereof”, is intended for a human or non-human mammal. Typically the patient is affected or likely to be infected with Nipah virus.

As used herein, the term “Nipah virus” has its general meaning in the art and refers to a virus that is a member of the Paramyxoviridae family and is related to the Hendra virus (formerly called equine morbillivirus). The Nipah virus was initially isolated in 1999 upon examining samples from an outbreak of encephalitis and respiratory illness among adult men in Malaysia and Singapore (see, e.g., Chua et al., Lancet. Oct. 9, 1999; 354(9186):1257-9 and Paton et al., Lancet. Oct. 9, 1999; 354(9186):1253-6). The host for Nipah virus is still unknown, but flying foxes (bats of the Pteropus genus) are suspected to be the natural host. The Nipah virus comprises a 6-gene, 18.2-kb, negative-sense single-stranded RNA (ssRNA) genome, which encodes 9 proteins: nucleoprotein (N), phosphoprotein (P), the interferon antagonists W and V, the viral C protein, a matrix protein (M), viral fusion and glycoproteins (F and G, respectively), and a large polymerase (L).

As used herein, the terms “polypeptide”, “peptide”, and “protein” are used interchangeably herein to refer to polymers of amino acids of any length. The terms also encompass an amino acid polymer that has been modified; for example, disulfide bond formation, glycosylation, lipidation, phosphorylation, or conjugation with a labeling component. Polypeptides when discussed in the context of gene therapy refer to the respective intact polypeptide, or any fragment or genetically engineered derivative thereof, which retains the desired biochemical function of the intact protein.

As used herein, the term “G protein” refers to the Nipah virus glycoprotein G. The G protein has a globular head domain formed of a six-bladed beta sheet-propeller, connected via a flexible stalk domain to a transmembrane anchor. The G protein binds to the cellular receptors ephrin B2 and ephrin B3, mediating viral attachment. Following attachment Nipah Virus glycoprotein G undergoes a conformational change that leads to triggering of glycoprotein F which leads to membrane fusion. An exemplary amino acid sequence for the G protein is represented by SEQ ID NO:1.

>sp|Q9IH62|GLYCP NIPAV Glycoprotein G
OS = Nipah virus OX = 121791 GN = G
PE = 1 SV = 1
SEQ ID NO: 1
MPAENKKVRFENTTSDKGKIPSKVIKSYYGTMDIKKINEGLLDSKILSAF
NTVIALLGSIVIIVMNIMIIQNYTRSTDNQAVIKDALQGIQQQIKGLADK
IGTEIGPKVSLIDTSSTITIPANIGLLGSKISQSTASINENVNEKCKFTL
PPLKIHECNISCPNPLPFREYRPQTEGVSNLVGLPNNICLQKTSNQILKP
KLISYTLPVVGQSGTCITDPLLAMDEGYFAYSHLERIGSCSRGVSKQRII
GVGEVLDRGDEVPSLFMTNVWTPPNPNTVYHCSAVYNNEFYYVLCAVSTV
GDPILNSTYWSGSLMMTRLAVKPKSNGGGYNQHQLALRSIEKGRYDKVMP
YGPSGIKQGDTLYFPAVGFLVRTEFKYNDSNCPITKCQYSKPENCRLSMG
IRPNSHYILRSGLLKYNLSDGENPKVVFIEISDQRLSIGSPSKIYDSLGQ
PVFYQASFSWDTMIKFGDVLTVNPLVVNWRNNTVISRPGQSQCPRENTCP
EICWEGVYNDAFLIDRINWISAGVELDSNQTAENPVFTVEKDNEILYRAQ
LASEDTNAQKTITNCFLLKNKIWCISLVEIYDTGDNVIRPKLFAVKIPEQ
CT

As used herein, the term “F protein” refers to the Nipah virus glycoprotein F. Nipah virus glycoprotein F is a class I fusion protein, with typical structural features. These include heptad repeats and a hydrophobic fusion peptide that bind to each other, forming a six-helix bundle which functions in membrane fusion processes. Nipah Virus attaches to target cells via glycoprotein G, which then undergoes a conformational change leading to triggering of Nipah virus glycoprotein F which leads to membrane fusion. An exemplary amino acid sequence for the F protein is represented by SEQ ID NO:2.

>sp|Q9IH63|FUS NIPAV Fusion glycoprotein FO
OS = Nipah virus OX = 121791 GN = F PE = 1
SV = 1
SEQ ID NO: 2
MVVILDKRCYCNLLILILMISECSVGILHYEKLSKIGLVKGVTRKYKIKS
NPLTKDIVIKMIPNVSNMSQCTGSVMENYKTRLNGILTPIKGALEIYKNN
THDLVGDVRLAGVIMAGVAIGIATAAQITAGVALYEAMKNADNINKLKSS
IESTNEAVVKLQETAEKTVYVLTALQDYINTNLVPTIDKISCKQTELSLD
LALSKYLSDLLFVFGPNLQDPVSNSMTIQAISQAFGGNYETLLRTLGYAT
EDFDDLLESDSITGQIIYVDLSSYYIIVRVYFPILTEIQQAYIQELLPVS
FNNDNSEWISIVPNFILVRNTLISNIEIGFCLITKRSVICNQDYATPMTN
NMRECLTGSTEKCPRELVVSSHVPRFALSNGVLFANCISVTCQCQTTGRA
ISQSGEQTLLMIDNTTCPTAVLGNVIISLGKYLGSVNYNSEGIAIGPPVF
TDKVDISSQISSMNQSLQQSKDYIKEAQRLLDTVNPSLISMLSMIILYVL
SIASLCIGLITFISFIIVEKKRNTYSRLEDRRVRPTSSGDLYYIGT

As used herein, the term “N protein” refers to the Nipah virus nucleoprotein that encapsidates the genome protecting it from nucleases. The encapsidated genomic RNA is termed the nucleocapsid (NC) and serves as template for transcription and replication. An exemplary amino acid sequence for the N protein is represented by SEQ ID NO:3.

>sp|Q9IK92|NCAP NIPAV Nucleoprotein OS =
Nipah virus OX = 121791 GN = N PE = 1
SV = 1
SEQ ID NO: 3
MSDIFEEAASFRSYQSKLGRDGRASAATATLTTKIRIFVPATNSPELRWE
LTLFALDVIRSPSAAESMKVGAAFTLISMYSERPGALIRSLLNDPDIEAV
IIDVGSMVNGIPVMERRGDKAQEEMEGLMRILKTARDSSKGKTPFVDSRA
YGLRITDMSTLVSAVITIEAQIWILIAKAVTAPDTAEESETRRWAKYVQQ
KRVNPFFALTQQWLTEMRNLLSQSLSVRKEMVEILIEVKKGGSAKGRAVE
IISDIGNYVEETGMAGFFATIREGLETRYPALALNEFQSDINTIKSLMLL
YREIGPRAPYMVLLEESIQTKFAPGGYPLLWSFAMGVATTIDRSMGALNI
NRGYLEPMYFRLGQKSARHHAGGIDQNMANRLGLSSDQVAELAAAVQETS
AGRQESNVQAREAKFAAGGVLIGGSDQDIDEGEEPIEQSGRQSVTFKREM
SISSLANSVPSSSVSTSGGTRLTNSLLNLRSRLAAKAAKEAASSNATDDP
AISNRTQGESEKKNNQDLKPAQNDLDEVRADV

As used herein, the term “polynucleotide” refers to a polymeric form of nucleotides of any length, including deoxyribonucleotides or ribonucleotides, or analogs thereof. A polynucleotide may comprise modified nucleotides, such as methylated nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment of the invention described herein that is a polynucleotide encompasses both the double-stranded form and each of two complementary single-stranded forms known or predicted to make up the double-stranded form.

As used herein, the expression “derived from” refers to a process whereby a first component (e.g., a first polypeptide), or information from that first component, is used to isolate, derive or make a different second component (e.g., a second polypeptide that is different from the first one).

As used herein, the term “encoding” refers to the inherent property of specific sequences of nucleotides in a polynucleotide, such as, for example, a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. The phrase “nucleotide sequence that encodes a protein or a RNA” may also include introns to the extent that the nucleotide sequence encoding the protein may in some version contain an intron(s).

As used herein, the terms “vector”, “cloning vector” and “expression vector” mean the vehicle by which a DNA or RNA sequence (e.g., a foreign gene) can be introduced into a host cell, so as to transform the host and promote expression (e.g., transcription and translation) of the introduced sequence.

As used herein, the term “promoter/regulatory sequence” refers to a polynucleotide sequence (such as, for example, a DNA sequence) recognized by the synthetic machinery of the cell, or introduced synthetic machinery, required to initiate the specific transcription of a polynucleotide sequence, thereby allowing the expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

As used herein, the term “operably linked” or “transcriptional control” refers to functional linkage between a regulatory sequence and a heterologous polynucleotide sequence resulting in expression of the latter. For example, a first polynucleotide sequence is operably linked with a second polynucleotide sequence when the first polynucleotide sequence is placed in a functional relationship with the second polynucleotide sequence. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame.

As used herein, the term “transformation” means the introduction of a “foreign” (i.e., extrinsic or extracellular) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA bas been “transformed”.

As used herein, the term “expression system” means a host cell and compatible vector under suitable conditions, e.g., for the expression of a protein coded for by foreign DNA carried by the vector and introduced to the host cell.

As used herein, the “percent identity” between the two sequences is a function of the number of identical positions shared by the sequences (i.e., % identity=number of identical positions/total number of positions×100), taking into account the number of gaps, and the length of each gap, which need to be introduced for optimal alignment of the two sequences. The comparison of sequences and determination of percent identity between two sequences can be accomplished using a mathematical algorithm, as described below. The percent identity between two amino acid sequences can be determined using the Needleman and Wunsch algorithm (Needleman, Saul B. & Wunsch, Christian D. (1970). “A general method applicable to the search for similarities in the amino acid sequence of two proteins”. Journal of Molecular Biology. 48 (3): 443-53.). The percent identity between two nucleotide or amino acid sequences may also be determined using for example algorithms such as EMBOSS Needle (pair wise alignment; available at www.ebi.ac.uk). For example, EMBOSS Needle may be used with a BLOSUM62 matrix, a “gap open penalty” of 10, a “gap extend penalty” of 0.5, a false “end gap penalty”, an “end gap open penalty” of 10 and an “end gap extend penalty” of 0.5. In general, the “percent identity” is a function of the number of matching positions divided by the number of positions compared and multiplied by 100. For instance, if 6 out of 10 sequence positions are identical between the two compared sequences after alignment, then the identity is 60%. The % identity is typically determined over the whole length of the query sequence on which the analysis is performed. Two molecules having the same primary amino acid sequence or polynucleotide sequence are identical irrespective of any chemical and/or biological modification. According to the invention a first amino acid sequence having at least 80% of identity with a second amino acid sequence means that the first sequence has 80; 81; 82; 83; 84; 85; 86; 87; 88; 89; 90; 91; 92; 93; 94; 95; 96; 97; 98; 99 or 100% of identity with the second amino acid sequence.

As used herein, the term “conjugate” or interchangeably “conjugated polypeptide” is intended to indicate a composite or chimeric molecule formed by the covalent attachment of one or more polypeptides. The term “covalent attachment” “or “conjugation” means that the polypeptide and the non-peptide moiety are either directly covalently joined to one another, or else are indirectly covalently joined to one another through an intervening moiety or moieties, such as a bridge, spacer, or linkage moiety or moieties. A particular conjugate is a fusion protein.

As used herein, the term “fusion protein” indicates a protein created through the attaching of two or more polypeptides which originated from separate proteins. In particular fusion proteins can be created by recombinant DNA technology and are typically used in biological research or therapeutics. Fusion proteins can also be created through chemical covalent conjugation with or without a linker between the polypeptides portion of the fusion proteins. In the fusion protein the two or more polypeptide are fused directly or via a linker.

As used herein, the term “directly” means that the first amino acid at the N-terminal end of a first polypeptide is fused to the last amino acid at the C-terminal end of a second polypeptide. This direct fusion can occur naturally as described in (Vigneron et al., Science 2004, PMID 15001714), (Warren et al., Science 2006, PMID 16960008), (Berkers et al., J. Immunol. 2015a, PMID 26401000), (Berkers et al., J. Immunol. 2015b, PMID 26401003), (Delong et al., Science 2016, PMID 26912858) (Liepe et al., Science 2016, PMID 27846572), (Babon et al., Nat. Med. 2016, PMID 27798614).

As used herein, the term “linker” has its general meaning in the art and refers to an amino acid sequence of a length sufficient to ensure that the proteins form proper secondary and tertiary structures. In some embodiments, the linker is a peptidic linker which comprises at least one, but less than 30 amino acids e.g., a peptidic linker of 2-30 amino acids, preferably of 10-30 amino acids, more preferably of 15-30 amino acids, still more preferably of 19-27 amino acids, most preferably of 20-26 amino acids. In some embodiments, the linker has 2; 3; 4; 5; 6; 7; 8; 9; 10; 11; 12; 13; 14; 15; 16; 17; 18; 19; 20; 21; 22; 23; 24; 25; 26; 27; 28; 29; 30 amino acid residues. Typically, linkers are those which allow the compound to adopt a proper conformation. The most suitable linker sequences (1) will adopt a flexible extended conformation, (2) will not exhibit a propensity for developing ordered secondary structure which could interact with the functional domains of fusion proteins, and (3) will have minimal hydrophobic or charged character which could promote interaction with the functional protein domains.

As used herein, the term “antibody” refers to immunoglobulin molecules and immunologically active portions of immunoglobulin molecules, i.e., molecules that contain an antigen binding site that immunospecifically binds to an antigen. In natural antibodies of rodents and primates, two heavy chains are linked to each other by disulfide bonds, and each heavy chain is linked to a light chain by a disulfide bond. There are two types of light chains, lambda (l) and kappa (k).

There are five main heavy chain classes (or isotypes) which determine the functional activity of an antibody molecule: IgM, IgD, IgG, IgA and IgE. Each chain contains distinct sequence domains. In typical IgG antibodies, the light chain includes two domains, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from non-hypervariable or framework regions (FR) can participate in the antibody binding site, or influence the overall domain structure and hence the combining site. Complementarity Determining Regions or CDRs refer to amino acid sequences that together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, typically includes six CDRs, comprising the CDRs set from each of a heavy and a light chain V region. Framework Regions (FRs) refer to amino acid sequences interposed between CDRs. Accordingly, the variable regions of the light and heavy chains typically comprise 4 framework regions and 3 CDRs of the following sequence: FR1-CDR1-FR2-CDR2-FR3-CDR3-FR4. The residues in antibody variable domains are conventionally numbered according to a system devised by Kabat et al. This system is set forth in Kabat et al., 1987, in Sequences of Proteins of Immunological Interest, US Department of Health and Human Services, NIH, USA (Kabat et al., 1992, hereafter “Kabat et al.”). The Kabat residue designations do not always correspond directly with the linear numbering of the amino acid residues in SEQ ID sequences. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence. The CDRs of the heavy chain variable domain are located at residues 31-35 (H-CDR1), residues 50-65 (H-CDR2) and residues 95-102 (H-CDR3) according to the Kabat numbering system. The CDRs of the light chain variable domain are located at residues 24-34 (L-CDR1), residues 50-56 (L-CDR2) and residues 89-97 (L-CDR3) according to the Kabat numbering system. For the agonist antibodies described hereafter, the CDRs have been determined using CDR finding algorithms from www.bioinf.org.uk—see the section entitled «How to identify the CDRs by looking at a sequence within the Antibodies pages.

As used herein, the term “immunoglobulin domain” refers to a globular region of an antibody chain (such as e.g. a chain of a heavy chain antibody or a light chain), or to a polypeptide that essentially consists of such a globular region.

As used herein, the term “Fc region” is used to define the C-terminal region of an immunoglobulin heavy chain, including native sequence Fc region and variant Fc regions. The human IgG heavy chain Fc region is generally defined as comprising the amino acid residue from position C226 or from P230 to the carboxyl-terminus of the IgG antibody. The numbering of residues in the Fc region is that of the EU index of Kabat. The C-terminal lysine (residue K447) of the Fc region may be removed, for example, during production or purification of the antibody. Accordingly, a composition of antibodies of the invention may comprise antibody populations with all K447 residues removed, antibody populations with no K447 residues removed, and antibody populations having a mixture of antibodies with and without the K447 residue.

As used herein, the term “chimeric antibody” refers to an antibody which comprises a VH domain and a VL domain of a non-human antibody, and a CH domain and a CL domain of a human antibody. In one embodiment, a “chimeric antibody” is an antibody molecule in which (a) the constant region (i.e., the heavy and/or light chain), or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, an agonist molecule, e.g., CD40 Ligand, hormone, growth factor, drug, etc.; or (b) the variable region, or a portion thereof, is altered, replaced or exchanged with a variable region having a different or altered antigen specificity. Chimeric antibodies also include primatized and in particular humanized antibodies. Furthermore, chimeric antibodies may comprise residues that are not found in the recipient antibody or in the donor antibody. These modifications are made to further refine antibody performance. For further details, see Jones et al., Nature 321:522-525 (1986); Riechmann et al., Nature 332:323-329 (1988); and Presta, Curr. Op. Struct. Biol. 2:593-596 (1992). (see U.S. Pat. No. 4,816,567; and Morrison et al., Proc. Natl. Acad. Sci. USA, 81:6851-6855 (1984)).

As used herein, the term “humanized antibody” include antibodies which have the 6 CDRs of a murine antibody, but humanized framework and constant regions. More specifically, the term “humanized antibody”, as used herein, may include antibodies in which CDR sequences derived from the germline of another mammalian species, such as a mouse, have been grafted onto human framework sequences.

As used herein the term “human monoclonal antibody”, is intended to include antibodies having variable and constant regions derived from human immunoglobulin sequences. The human antibodies of the present invention may include amino acid residues not encoded by human immunoglobulin sequences (e.g., mutations introduced by random or site-specific mutagenesis in vitro or by somatic mutation in vivo). However, in one embodiment, the term “human monoclonal antibody”, as used herein, is not intended to include antibodies in which CDR sequences derived from the germline of another mammalian species, such as a mouse, have been grafted onto human framework sequences.

As used herein, the term “immune response” refers to a reaction of the immune system to an antigen in the body of a host, which includes generation of an antigen-specific antibody and/or cellular cytotoxic response. The immune response to an initial antigenic exposure (primary immune response) is typically, detectable after a lag period of from several days to two weeks; the immune response to subsequent stimulus (secondary immune response) by the same antigen is more rapid than in the case of the primary immune response. An immune response to a transgene product may include both humoral (e.g., antibody response) and cellular (e.g., cytolytic T cell response) immune responses that may be elicited to an immunogenic product encoded by the transgene. The level of the immune response can be measured by methods known in the art (e.g., by measuring antibody titre).

As used herein the term “APCs” or “Antigen Presenting Cells” denotes cells that are capable of activating T-cells, and include, but are not limited to, certain macrophages, B cells and dendritic cells.

As used herein, the term “Dendritic cells” or “DCs” refer to any member of a diverse population of morphologically similar cell types found in lymphoid or non-lymphoid tissues. These cells are characterized by their distinctive morphology, high levels of surface MHC-class II expression (Steinman, et al., Ann. Rev. Immunol. 9:271 (1991); incorporated herein by reference for its description of such cells).

As used herein, the term “CD40” has its general meaning in the art and refers to human CD40 polypeptide receptor. In some embodiments, CD40 is the isoform of the human canonical sequence as reported by UniProtKB-P25942 (also referred as human TNR5).

As used herein, the term “CD40L” has its general meaning in the art and refers to human CD40L polypeptide, for example, as reported by UniProtKB-P25942, including its CD40-binding domain of SEQ ID NO:4. CD40L may be expressed as a soluble polypeptide and is the natural ligand of CD40 receptor.

>CD40L binding domain
SEQ ID NO: 4
MQKGDQNPQIAAHVISEASSKTTSVLQWAEKGYYTMSNNLVTLENGKQLT
VKRQGLYYIYAQVTFCSNREASSQAPFIASLCLKSPGRFERILLRAANTH
SSAKPCGQQSIHLGGVFELQPGASVFVNVTDPSQVSHGTGFTSFGLLKL

As used herein, the term “CD40 agonist antibody” is intended to refer to an antibody that increases CD40 mediated signaling activity in the absence of CD40L in a cell-based assay, such as the B cell proliferation assay. In particular, the CD40 agonist antibody (i) it induces the proliferation of B cell, as measured in vitro by flow cytometric analysis, or by analysis of replicative dilution of CFSE-labeled cells; and/or (ii) induces the secretion of cytokines, such as IL-6, IL-12, or IL-15, as measured in vitro with a dendritic cell activation assay.

As used herein, the term “Langerin” has its general meaning in the art and refers to human C-type lectin domain family 4 member K polypeptide. In some embodiments, Langerin is the isoform of the human canonical sequence as reported by UniProtKB-Q9UJ71 (also referred as human CD207).

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular interval, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the term “pharmaceutical composition” refers to a composition described herein, or pharmaceutically acceptable salts thereof, with other agents such as carriers and/or excipients. The pharmaceutical compositions as provided herewith typically include a pharmaceutically acceptable carrier.

As used herein, the term “pharmaceutically acceptable carrier” includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical-Sciences, Sixteenth Edition, E. W. Martin (Mack Publishing Co., Easton, Pa., 1980) discloses various carriers used in formulating pharmaceutical compositions and known techniques for the preparation thereof.

As used herein, the term “vaccination” or “vaccinating” means, but is not limited to, a process to elicit an immune response in a subject against a particular antigen.

As used herein, the term “vaccine composition” is intended to mean a composition which can be administered to humans or to animals in order to induce an immune system response; this immune system response can result in the activation of certain cells, in particular APCs, T lymphocytes and B lymphocytes.

As used herein the term “antigen” refers to a molecule capable of being specifically bound by an antibody or by a T cell receptor (TCR) if processed and presented by MHC molecules. An antigen is additionally capable of being recognized by the immune system and/or being capable of inducing a humoral immune response and/or cellular immune response leading to the activation of B- and/or T-lymphocytes. An antigen can have one or more epitopes or antigenic sites (B- and T-epitopes).

As used herein, the term “adjuvant” refers to a compound that can induce and/or enhance the immune response against an antigen when administered to a subject or an animal. It is also intended to mean a substance that acts generally to accelerate, prolong, or enhance the quality of specific immune responses to a specific antigen. In the context of the present invention, the term “adjuvant” means a compound, which enhances both innate immune response by affecting the transient reaction of the innate immune response and the more long-lived effects of the adaptive immune response by activation and maturation of the antigen-presenting cells (APCs) especially Dendritic cells (DCs).

As used herein, the expression “therapeutically effective amount” is meant a sufficient amount of the active ingredient of the present invention to induce an immune response at a reasonable benefit/risk ratio applicable to the medical treatment.

### Antibodies of the Present Invention:

The first object of the present invention relates to an antibody that is directed against a surface antigen of an antigen presenting cell wherein the heavy chain and/or the light chain is conjugated or fused to a polypeptide having at least 80% of identity with the amino acid sequence that ranges from the amino acid residue (Q) at position 71 to the amino acid residue (T) at position 602 in SEQ ID NO:1 “(Niv(G)_{B} ectodomain”).

In some embodiments, the light chain of the antibody is conjugated or fused to the polypeptide having at least 80% of identity with the amino acid sequence that ranges from the amino acid residue (Q) at position 71 to the amino acid residue (T) at position 602 in SEQ ID NO:1 (Niv(G)_{B} ectodomain).

In some embodiments, the heavy chain of the antibody is conjugated or fused to the polypeptide having at least 80% of identity with the amino acid sequence that ranges from the amino acid residue (Q) at position 71 to the amino acid residue (T) at position 602 in SEQ ID NO:1 (Niv(G)_{B} ectodomain).

The heavy chain and/or the light chain of the antibody is conjugated or fused to the Niv(G)_{B} ectodomain via its C-terminal. In some embodiments, the heavy chain and/or the light chain of the antibody is fused to the N-terminal of the Niv(G)_{B} ectodomain.

In some embodiments, the heavy chain and/or the light chain of the antibody is conjugated to the Niv(G)_{B} ectodomain by using chemical coupling. Several methods are known in the art for the attachment or conjugation of an antibody to its conjugate moiety. Examples of linker types that have been used to conjugate a moiety to an antibody include, but are not limited to, hydrazones, thioethers, esters, disulfides and peptide-containing linkers, such as valine-citruline linker. A linker can be chosen that is, for example, susceptible to cleavage by low pH within the lysosomal compartment or susceptible to cleavage by proteases, such as proteases preferentially expressed in tumor tissue such as cathepsins (e.g., cathepsins B, C, D). Techniques for conjugating polypeptides and in particular, are well-known in the art (See, e.g., Arnon et al., “Monoclonal Antibodies For Immunotargeting Of Drugs In Cancer Therapy,” in Monoclonal Antibodies And Cancer Therapy (Reisfeld et al. eds., Alan R. Liss, Inc., 1985); Hellstrom et al., “Antibodies For Drug Delivery,” in Controlled Drug Delivery (Robinson et al. eds., Marcel Deiker, Inc., 2nd ed. 1987); Thorpe, “Antibody Carriers Of Cytotoxic Agents In Cancer Therapy: A Review,” in Monoclonal Antibodies '84: Biological And Clinical Applications (Pinchera et al. eds., 1985); “Analysis, Results, and Future Prospective of the Therapeutic Use of Radiolabeled Antibody In Cancer Therapy,” in Monoclonal Antibodies For Cancer Detection And Therapy (Baldwin et al. eds., Academic Press, 1985); and Thorpe et al., 1982, Immunol. Rev. 62:119-58; see also, e.g., PCT publication WO 89/12624.) Typically, the peptide is covalently attached to lysine or cysteine residues on the antibody, through N-hydroxysuccinimide ester or maleimide functionality respectively. Methods of conjugation using engineered cysteines or incorporation of unnatural amino acids have been reported to improve the homogeneity of the conjugate (Axup, J. Y., Bajjuri, K. M., Ritland, M, Hutchins, B. M, Kim, C. H, Kazane, S. A., Halder, R., Forsyth, J S., Santidrian, A F, Stafin, K., et al. (2012). Synthesis of site-specific antibody-drug conjugates using unnatural amino acids. Proc. Natl. Acad. Sci. USA 109, 16101-16106; Junutula, J. R., Flagella, KM, Graham, R. A., Parsons, K. L., Ha, E., Raab, H., Bhakta, S., Nguyen, T, Dugger, D. L., Li, G., et al. (2010). Engineered thio-trastuzumab-DM1 conjugate with an improved therapeutic index to target human epidermal growth factor receptor 2-positive breast cancer. Clin. Cancer Res. 16, 4769-4778). Junutula et al. (Nat Biotechnol. 2008; 26:925-32) developed cysteine-based site-specific conjugation called “THIOMABs” (TDCs) that are claimed to display an improved therapeutic index as compared to conventional conjugation methods. Conjugation to unnatural amino acids that have been incorporated into the antibody is also being explored for ADCs; however, the generality of this approach is yet to be established (Axup et al., 2012). In particular the one skilled in the art can also envisage Fc-containing polypeptide engineered with an acyl donor glutamine-containing tag (e.g., Gin-containing peptide tags or Q-tags) or an endogenous glutamine that are made reactive by polypeptide engineering (e.g., via amino acid deletion, insertion, substitution, or mutation on the polypeptide). Then a transglutaminase can covalently crosslink with an amine donor agent (e.g., a small molecule comprising or attached to a reactive amine) to form a stable and homogenous population of an engineered Fc-containing polypeptide conjugate with the amine donor agent being site-specifically conjugated to the Fc-containing polypeptide through the acyl donor glutamine-containing tag or the accessible/exposed/reactive endogenous glutamine (WO 2012059882).

In some embodiments, the heavy chain and/or the light chain of the antibody is conjugated to the Niv(G)_{B} ectodomain by a dockerin domain or multiple domains to permit non-covalent coupling to cohesin fusion proteins as described in US20160031988A1 and US20120039916A1.

In some embodiments, the heavy chain and/or light of the antibody is fused to the Niv(G)_{B} ectodomain to form a fusion protein.

In some embodiments, the Niv(G)_{B} ectodomain is fused either directly or via a linker to the heavy and/or light chain. As used herein, the term “directly” means that the first amino acid at the N-terminal end of the Niv(G)_{B} ectodomain is fused to the last amino acid at the C-terminal end of the heavy or light chain. This direct fusion can occur naturally as described in (Vigneron et al., Science 2004, PMID 15001714), (Warren et al., Science 2006, PMID 16960008), (Berkers et al., J. Immunol. 2015a, PMID 26401000), (Berkers et al., J. Immunol. 2015b, PMID 26401003), (Delong et al., Science 2016, PMID 26912858) (Liepe et al., Science 2016, PMID 27846572), (Babon et al., Nat. Med. 2016, PMID 27798614).

In some embodiments, the N-terminal of the Niv(G)_{B} ectodomain is fused to the C-terminal of the heavy chain directly or via a linker.

In some embodiments, the linker is selected from the group consisting of SEQ ID NO:5 (FlexV1), SEQ ID NO:6 (f1), SEQ ID NO:7 (f2), SEQ ID NO:8 (f), or SEQ ID NO:9 (f4), as described below.

| | flexV1 |
|---|---|
| (SEQ ID NO: 5) | |
| | QTPTNTISVTPTNNSTPTNNSNPKPNP, |
| | f1 |
| (SEQ ID NO: 6) | |
| | SSVSPTTSVHPTPTSVPPTPTKSSP, |
| | f2 |
| (SEQ ID NO: 7) | |
| | PTSTPADSSTITPTATPTATPTIKG, |
| | f3 |
| (SEQ ID NO: 8) | |
| | TVTPTATATPSAIVTTITPTATTKP, |
| | f4 |
| (SEQ ID NO: 9) | |
| | TNGSITVAATAPTVTPTVNATPSAA, |

In some embodiments, the antibody of the present invention comprises:
a heavy chain of the antibody that is conjugated or fused to the polypeptide having at least 80% of identity with the amino acid sequence that ranges from the amino acid residue (Q) at position 71 to the amino acid residue (T) at position 602 in SEQ ID NO:1 (Niv(G)_{B} ectodomain) and
a light chain is conjugated or fused to the polypeptide having at least 80% of identity with the amino acid sequence that ranges from the amino acid residue (K) at position 45 to the amino acid residue (I) at position 90 in SEQ ID NO:2 (“predicted epitope-rich peptide Niv(F)_{B}”).

In some embodiments, the C-terminal of the light chain of the antibody is conjugated or fused to the N-terminal of the the polypeptide having at least 80% of identity with the amino acid sequence that ranges from the amino acid residue (K) at position 45 to the amino acid residue (I) at position 90 in SEQ ID NO:2 (“predicted epitope-rich peptide Niv(F)_{B}”), and the C-terminal of the heavy chain of the antibody is conjugated or fused to the N-terminal of the polypeptide having at least 80% of identity with the amino acid sequence that ranges from the amino acid residue (Q) at position 71 to the amino acid residue (T) at position 602 in SEQ ID NO:1 (Niv(G)_{B} ectodomain).

In some embodiments, the antibody of the present invention comprises:
a heavy chain of the antibody that is conjugated or fused to the polypeptide having at least 80% of identity with the amino acid sequence that ranges from the amino acid residue (Q) at position 71 to the amino acid residue (T) at position 602 in SEQ ID NO:1 (Niv(G)_{B} ectodomain) and
a light chain is conjugated or fused to the polypeptide having at least 80% of identity with the amino acid sequence that ranges from the amino acid residue (I) at position 318 to the amino acid residue (L) at position 355 in SEQ ID NO:3 (“predicted epitope-rich peptide Niv(N)_{B}”).

In some embodiments, the C-terminal of the light chain of the antibody is conjugated or fused to the N-terminal of the polypeptide having at least 80% of identity with the amino acid sequence that ranges from the amino acid residue (I) at position 318 to the amino acid residue (L) at position 355 in SEQ ID NO:3 (“predicted epitope-rich peptide Niv(N)_{B}”) and the C-terminal of the heavy chain of the antibody is conjugated or fused to the N-terminal of the polypeptide having at least 80% of identity with the amino acid sequence that ranges from the amino acid residue (Q) at position 71 to the amino acid residue (T) at position 602 in SEQ ID NO:1 (Niv(G)_{B} ectodomain).

In some embodiments, the antibody of the present invention comprises:
a heavy chain of the antibody that is conjugated or fused to the polypeptide having at least 80% of identity with the amino acid sequence that ranges from the amino acid residue (Q) at position 71 to the amino acid residue (T) at position 602 in SEQ ID NO:1 (Niv(G)_{B} ectodomain) and
a light chain is conjugated or fused to both i) the polypeptide having at least 80% of identity with the amino acid sequence that ranges from the amino acid residue (K) at position 45 to the amino acid residue (I) at position 90 in SEQ ID NO:2 (“predicted epitope-rich peptide Niv(F)_{B}”) and ii) the polypeptide having at least 80% of identity with the amino acid sequence that ranges from the amino acid residue (I) at position 318 to the amino acid residue (L) at position 355 in SEQ ID NO:3 (“predicted epitope-rich peptide Niv(N)_{B}”).

In some embodiments, the antibody of the present invention comprises:
a heavy chain of the antibody that is conjugated or fused to the polypeptide having at least 80% of identity with the amino acid sequence that ranges from the amino acid residue (Q) at position 71 to the amino acid residue (T) at position 602 in SEQ ID NO:1 (Niv(G)_{B} ectodomain) and
a light chain is conjugated or fused to both a fusion protein wherein i) the polypeptide having at least 80% of identity with the amino acid sequence that ranges from the amino acid residue (I) at position 318 to the amino acid residue (L) at position 355 in SEQ ID NO:3 (“predicted epitope-rich peptide Niv(N)_{B}”) is fused to ii) the polypeptide having at least 80% of identity with the amino acid sequence that ranges from the amino acid residue (K) at position 45 to the amino acid residue (I) at position 90 in SEQ ID NO:2 (“predicted epitope-rich peptide Niv(F)_{B}”).

In some embodiments, the light chain is conjugated or fused to a fusion protein comprising the polypeptide having at least 80% of identity with the amino acid sequence that ranges from the amino acid residue (K) at position 45 to the amino acid residue (I) at position 90 in SEQ ID NO:2 (“predicted epitope-rich peptide Niv(F)_{B}”) fused via its C-terminal directly or via a linker to the N-terminal of the polypeptide having at least 80% of identity with the amino acid sequence that ranges from the amino acid residue (I) at position 318 to the amino acid residue (L) at position 355 in SEQ ID NO:3 (“predicted epitope-rich peptide Niv(N)_{B}”).

In some embodiments, the light chain of the antibody is conjugated or fused to the fusion protein via its C-terminal.

In some embodiments, the polypeptide having at least 80% of identity with the amino acid sequence that ranges from the amino acid residue (I) at position 318 to the amino acid residue (L) at position 355 in SEQ ID NO:3 (“predicted epitope-rich peptide Niv(N)_{B}”) is fused via a linker to the polypeptide having at least 80% of identity with the amino acid sequence that ranges from the amino acid residue (K) at position 45 to the amino acid residue (I) at position 90 in SEQ ID NO:2 (“predicted epitope-rich peptide Niv(F)_{B}”).

In some embodiments, the linker consists of the amino acid sequence as set forth in SEQ ID NO:10.

| SEQ ID NO: 10 | |
|---|---|
| | AEAAAKEAAAKA |

In some embodiments, the antibody of the present invention comprises:
a heavy chain of the antibody that is conjugated or fused to the polypeptide having at least 80% of identity with the amino acid sequence that ranges from the amino acid residue (Q) at position 71 to the amino acid residue (T) at position 602 in SEQ ID NO:1 (Niv(G)_{B} ectodomain) and
a light chain is conjugated or fused to both the fusion protein having at least 80% of identity with amino acid sequence as set forth in SEQ ID NO:11.

SEQ ID NO: 11
KYKIKSNPLTKDIVIKMIPNVSNMSQCTGSVMENYKTRLNGILTPIAEAA
AKEAAAKAIQTKFAPGGYPLLWSFAMGVATTIDRSMGALNINRGYL

In some embodiments, the antibody is an IgG antibody, preferably of an IgG1 or IgG4 antibody, or even more preferably of an IgG4 antibody.

In some embodiments, the antibody is a chimeric antibody, in particular a chimeric mouse/human antibody.

In some embodiments, the antibody is humanized antibody.

Chimeric or humanized antibodies can be prepared based on the sequence of a murine monoclonal antibody prepared as described above. DNA encoding the heavy and light chain immunoglobulins can be obtained from the murine hybridoma of interest and engineered to contain non-murine (e.g., human) immunoglobulin sequences using standard molecular biology techniques. For example, to create a chimeric antibody, the murine variable regions can be linked to human constant regions using methods known in the art (see e.g., U.S. Pat. No. 4,816,567 to Cabilly et al.). To create a humanized antibody, the murine CDR regions can be inserted into a human framework using methods known in the art. See e.g., U.S. Pat. No. 5,225,539 to Winter, and U.S. Pat. Nos. 5,530,101; 5,585,089; 5,693,762 and 6,180,370 to Queen et al.

In some embodiments, the antibody is a human antibody. In some embodiments, human antibodies can be identified using transgenic or transchromosomic mice carrying parts of the human immune system rather than the mouse system. These transgenic and transchromosomic mice include mice referred to herein as HuMAb mice and KM mice, respectively, and are collectively referred to herein as “human Ig mice.” The HuMAb Mouse® (Medarex, Inc.) contains human immunoglobulin gene miniloci that encode un-rearranged human heavy (μ and γ) and κ light chain immunoglobulin sequences, together with targeted mutations that inactivate the endogenous μ and κ chain loci (see e.g., Lonberg, et al., 1994 Nature 368(6474): 856-859). In another embodiment, human antibodies can be raised using a mouse that carries human immunoglobulin sequences on transgenes and transchomosomes such as a mouse that carries a human heavy chain transgene and a human light chain transchromosome. Such mice, referred to herein as “KM mice”, are described in detail in PCT Publication WO 02/43478 to Ishida et al.

In some embodiments, the antibody is specific for a cell surface marker of a professional APC. The antibody may be specific for a cell surface marker of another professional APC, such as a B cell or a macrophage.

In some embodiments, the antibody is selected from an antibody that specifically binds to DC immunoreceptor (DCIR), MHC class I, MHC class II, CD1, CD2, CD3, CD4, CD8, CD11b, CD14, CD15, CD16, CD19, CD20, CD29, CD31, CD40, CD43, CD44, CD45, CD54, CD56, CD57, CD58, CD83, CD86, CMRF-44, CMRF-56, DCIR, DC-ASPGR, CLEC-6, CD40, BDCA-2, MARCO, DEC-205, mannose receptor, Langerin, DECTIN-1, B7-1, B7-2, IFN-γ receptor and IL-2 receptor, ICAM-1, Fey receptor, LOX-1, and ASPGR.

In some embodiments, the antibody is specific for CD40.

In some embodiments, the anti-CD40 antibody derives from the 12E12 antibody and comprises:
a heavy chain comprising the complementarity determining regions CDR1H, CDR2H and CDR3H, the CDR1H having the amino acid sequence GFTFSDYYMY (SEQ ID NO:12), the CDR2H having the amino acid sequence YINSGGGSTYYPDTVKG (SEQ ID NO:13), and the CDR3H having the amino acid sequence RGLPFHAMDY (SEQ ID NO:14),
and a light chain comprising the complementarity determining regions CDR1L, CDR2L and CDR3L, the CDR1L having the amino acid sequence SASQGISNYLN (SEQ ID NO:15) the CDR2L having the amino acid sequence YTSILHS (SEQ ID NO:16) and the CDR3L having the amino acid sequence QQFNKLPPT (SEQ ID NO:17).

In some embodiments, the anti-CD40 antibody derives from the 11B6 antibody and comprises:
a heavy chain comprising the complementarity determining regions CDR1H, CDR2H and CDR3H, the CDR1H having the amino acid sequence GYSFTGYYMH (SEQ ID NO:18), the CDR2H having the amino acid sequence RINPYNGATSYNQNFKD (SEQ ID NO:19), and the CDR3H having the amino acid sequence EDYVY (SEQ ID NO:20), and
a light chain comprising the complementarity determining regions CDR1L, CDR2L and CDR3L, the CDR1L having the amino acid sequence RSSQSLVHSNGNTYLH (SEQ ID NO:21) the CDR2L having the amino acid sequence KVSNRFS (SEQ ID NO:22) and the CDR3L having the amino acid sequence SQSTHVPWT (SEQ ID NO:23).

In some embodiments, the anti-CD40 antibody derives from the 12B4 antibody and comprises:
a heavy chain comprising the complementarity determining regions CDR1H, CDR2H and CDR3H, the CDR1H having the amino acid sequence GYTFTDYVLH (SEQ ID NO:24), the CDR2H having the amino acid sequence YINPYNDGTKYNEKFKG (SEQ ID NO:25), and the CDR3H having the amino acid sequence GYPAYSGYAMDY (SEQ ID NO:26), and
alight chain comprising the complementarity determining regions CDR1L, CDR2L and CDR3L, the CDR1L having the amino acid sequence RASQDISNYLN (SEQ ID NO:27) the CDR2L having the amino acid sequence YTSRLHS (SEQ ID NO:28) and the CDR3L having the amino acid sequence HHGNTLPWT (SEQ ID NO:29).

In some embodiments, the anti-CD40 antibody is selected from the group consisting of selected mAb1, mAb2, mAb3, mAb4, mAb5 and mAb6 as described in Table A.

TABLE A
CD40 antibodies

| | | |
|---|---|---|
| mAb1 [11B6 | SEQ ID NO: 30 | SEQ ID NO: 31 |
| VH/VkV2] | | |
| mAb2 | SEQ ID NO: 32 | SEQ ID NO: 31 |
| [11B6 | | |
| VHV3/VkV2] | | |
| mAb3 | SEQ ID NO: 33 | SEQ ID NO: 34 |
| [12B4] | | |
| mAb4 | SEQ ID NO: 35 | SEQ ID NO: 36 |
| [24A3] | | |
| mAb5 | SEQ ID NO: 37 | SEQ ID NO: 38 |
| [CP870,893] | | |
| mAb 6 | SEQ ID NO: 39 | SEQ ID NO: 40 |
| [12E12] | | |

(Amino acid sequence of variable heavy chain
region (VH) (v2) of Humanized 11B6)
SEQ ID NO: 30
EVQLVQSGAEVKKPGASVKISCKASGYSFTGYYMHWVKQAHGQGLEWIGR
INPYNGATSYNQNFKDRATLTVDKSTSTAYMELSSLRSEDTAVYYCARED
YVYWGQGTTVTVSSAS
(Amino acid sequence of variable light chain
(VL) Vk (v2) of humanized 11B6 VL)
SEQ ID NO: 31
DVVMTQSPLSLPVTLGQPASISCRSSQSLVHSNGNTYLHWYQQRPGQSPR
LLIYKVSNRFSGVPDRESGSGSGTDFTLKISRVEAEDVGVYFCSQSTHVP
WTFGGGTK
(Amino acid sequence of variable heavy chain
region VH (v3) of humanized 11B6)
SEQ ID NO: 32
EVQLVQSGAEVKKPGASVKVSCKASGYSFTGYYMHWVRQAPGQGLEWIGR
INPYNGATSYNQNFKDRVTLTVDKSTSTAYMELSSLRSEDTAVYYCARED
YVYWGQGTTVTVSSAS
(VH amino acid sequence of mAb3 (12B4))
SEQ ID NO: 33
EVQLQQSGPELVKPGASVKMSCKASGYTFTDYVLHWVKQKPGQGLEWIGY
INPYNDGTKYNEKFKGKATLTSDKSSSTAYMELSSLTSEDSAVYYCARGY
PAYSGYAMDYWGQGTSVTVSSAS
(VL amino acid sequence of mAb3 (12B4))
SEQ ID NO: 34
DIQMTQTTSSLSASLGDRVTISCRASQDISNYLNWYQQKPDGTVKLLIYY
TSRLHSGVPSRFSGSGSGTDYSLTISNLEQEDIATYFCHHGNTLPWTFGG
GTK
(VH amino acid sequence of mAb4 (24A3 HC) )
SEQ ID NO: 35
DVQLQESGPDLVKPSQSLSLTCTVTGYSITSDYSWHWIRQFPGNKLEWMG
YIYYSGSTNYNPSLKSRISITRDTSKNQFFLQLNSVTTEDSATYFCARFY
YGYSFFDYWGQGTTLTVSSAS
(VL amino acid sequence of mAb4 (24A3 KC) )
SEQ ID NO: 36
QIVLTQSPAFMSASPGEKVTMTCSASSSVSYMHWYQQKSGTSPKRWIYDT
SKLASGVPARFSGSGSGTSYSLTISSMEAEDAATYYCQQWSSNPLTFGAG
TK
(VH amino acid sequence of mAb5)
SEQ ID NO: 37
QVQLVQSGAEVKKPGASVKVSCKASGYTFTGYYMHWVRQAPGQGLEWMGW
INPDSGGTNYAQKFQGRVTMTRDTSISTAYMELNRLRSDDTAVYYCARDQ
PLGYCTNGVCSYFDYWGQGTLVTVSSAS
(VL amino acid sequence of mAb5)
SEQ ID NO: 38
DIQMTQSPSSVSASVGDRVTITCRASQGIYSWLAWYQQKPGKAPNLLIYT
ASTLQSGVPSRESGSGSGTDFTLTISSLQPEDFATYYCQQANIFPLTFGG
GTK
(VH amino acid sequence of mAb6 (12E12 H3
Humanized HC))
SEQ ID NO: 39
EVQLVESGGGLVQPGGSLKLSCATSGFTFSDYYMYWVRQAPGKGLEWVAY
INSGGGSTYYPDTVKGRFTISRDNAKNTLYLQMNSLRAEDTAVYYCARRG
LPFHAMDYWGQGTLVTVSSAS
(VL amino acid sequence of mAb6 (Humanized K2
12E12))
SEQ ID NO: 40
DIQMTQSPSSLSASVGDRVTITCSASQGISNYLNWYQQKPGKAVKLLIYY
TSILHSGVPSRESGSGSGTDYTLTISSLQPEDFATYYCQQFNKLPPTFGG
GTK

In some embodiments, the anti-CD40 antibody comprises: a heavy chain wherein the variable domain has the the amino acid sequence set forth as SEQ ID NO:30, and a light chain wherein the variable domain has a sequence set forth as SEQ ID NO:31.

In some embodiments, the anti-CD40 antibody comprises: a heavy chain wherein the variable domain has the the amino acid sequence set forth as SEQ ID NO:32, and a light chain wherein the variable domain has a sequence set forth as SEQ ID NO:31.

In some embodiments, the anti-CD40 antibody comprises: a heavy chain wherein the variable domain has the the amino acid sequence set forth as SEQ ID NO:33, and a light chain wherein the variable domain has a sequence set forth as SEQ ID NO:34.

In some embodiments, the anti-CD40 antibody comprises: a heavy chain wherein the variable domain has the the amino acid sequence set forth as SEQ ID NO:35, and a light chain wherein the variable domain has a sequence set forth as SEQ ID NO:36.

In some embodiments, the anti-CD40 antibody comprises: a heavy chain wherein the variable domain has the the amino acid sequence set forth as SEQ ID NO:37, and a light chain wherein the variable domain has a sequence set forth as SEQ ID NO:38.

In some embodiments, the anti-CD40 antibody comprises: a heavy chain wherein the variable domain has the the amino acid sequence set forth as SEQ ID NO:39, and a light chain wherein the variable domain has a sequence set forth as SEQ ID NO:40.

In some embodiments, the anti-CD40 antibody is a CD40 agonist antibody. CD40 agonist antibodies are described in WO2010/009346, WO2010/104747 and WO2010/104749. Other anti-CD40 agonist antibodies in development include CP-870,893 that is a fully human IgG2 CD40 agonist antibody developed by Pfizer. It binds CD40 with a KD of 3.48×10-10 M, but does not block binding of CD40L (see e.g., U.S. Pat. No. 7,338,660) and SGN-40 that is a humanized IgG1 antibody developed by Seattle Genetics from mouse antibody clone S2C6, which was generated using a human bladder carcinoma cell line as the immunogen. It binds to CD40 with a KD of 1.0×10-9 M and works through enhancing the interaction between CD40 and CD40L, thus exhibiting a partial agonist effect (Francisco J A, et al., Cancer Res, 60: 3225-31, 2000). Even more particularly, the CD40 agonist antibody is selected from the group consisting of selected mAb1, mAb2, mAb3, mAb4, mAb5 and mAb6 as described in Table A.

In some embodiments, the antibody is specific for Langerin. In some embodiments, the antibody derives from the antibody 15B10 having ATCC Accession No. PTA-9852. In some embodiments, the antibody derives from the antibody 2G3 having ATCC Accession No. PTA-9853. In some embodiments, the antibody derives from the antibody 91E7, 37C1, or 4C7 as described in WO2011032161.

In some embodiments, the anti-Langerin antibody comprises a heavy chain comprising the complementarity determining regions CDR1H, CDR2H and CDR3H of the 15B10 antibody and a light chain comprising the complementarity determining regions CDR1L, CDR2L and CDR3L of the 15B10 antibody.

In some embodiments, the anti-Langerin antibody comprises a heavy chain comprising the complementarity determining regions CDR1H, CDR2H and CDR3H of the 2G3 antibody and a light chain comprising the complementarity determining regions CDR1L, CDR2L and CDR3L of the 2G3 antibody.

In some embodiments, the anti-Langerin antibody comprises a heavy chain comprising the complementarity determining regions CDR1H, CDR2H and CDR3H of the 4C7 antibody and a light chain comprising the complementarity determining regions CDR1L, CDR2L and CDR3L of the 4C7 antibody.

In some embodiments, the antibody is selected from the group consisting of selected mAb7, mAb8 and mAb9, as described in Table B.

| | | |
|---|---|---|
| mAb7 | SEQ ID NO: 41 | SEQ ID NO: 42 |
| [15B10] | | |
| mAb8 | SEQ ID NO: 43 | SEQ ID NO: 44 |
| [2G3] | | |
| mAb9 | SEQ ID NO: 45 | SEQ ID NO: 46 |
| [4C7] | | |

(Amino acid sequence of variable heavy chain
region (VH) of 15B10)
SEQ ID NO: 41
SVKMSCKASGYTFTDYVISWVKQRTGQGLEWIGDIYPGSGYSFYNENFKG
KATLTADKSSTTAYMQLSSLTSEDSAVYFCA
(Amino acid sequence of variable light chain
(VL) 15B10)
SEQ ID NO: 42
ASISCRSSQSLVHSNGNTYLHWYLQKPGQSPKLLIYKVSNRFSGVPDRES
GSGSGTNFTLKISRVEAEDLGLYFCS
(Amino acid sequence of variable heavy chain
region (VH) of 2G3)
SEQ ID NO: 43
SSVKMSCKASGYTFTDYVISWVKQRTGQGLEWIGDIYPGSGYSFYNENFK
GKATLTADKSSTTAYMQLSSLTSEDSAVYFCA
(Amino acid sequence of variable light chain
(VL) 2G3)
SEQ ID NO: 44
VTLTCRSSTGAVTTSNYANWVQEKPDHLFTGLIGGTNNRVSGVPARFSGS
LIGDKAALTITGAQTEDEAIYFCA
(Amino acid sequence of the heavy chain of 4C7)
SEQ ID NO: 45
QVQLQQSGAELVRPGASVTLSCKASGYTFIDHDMHWVQQTPVYGLEWIGA
IDPETGDTGYNQKFKGKAILTADKSSRTAYMELRSLTSEDSAVYYCTIPF
YYSNYSPFAYWGQGALVTVSAAKTTAPSVYPLAPVCGGTTGSSVTLGCLV
KGYFPEPVTLTWNSGSLSSGVHTFPALLQSGLYTLSSSVTVTSNTWPSQT
ITCNVAHPASSTKVDKKIEPRVPITQNPCPPLKECPPCADLLGGPSVFIF
PPKIKDVLMISLSPMVTCVVVDVSEDDPDAQISWFVNNVEVHTAQTQTHR
EDYNSTLRVVSALPIQHQDWMSGKEFKCKVNNRALPSPIEKTISKPRGPV
RAPQVYVLPPPAEEMTKKEFSLTCMITGFLPAEIAVDWTSNGRTEQNYKN
TATVLDSDGSYFMYSKLRVQKSTWERGSLFACSVVHEGLHNHLTTKTISR
SLGKAS
(Amino acid sequence of light chain of 4C7)
SEQ ID NO: 46
QIVLSQSPAILSASPGEKVTMTCRASSSVSYMHWYQRKPGSSPKPWIYAT
SNLASGVPARFSGSGSGTSYSLTISRVEAEDAATYYCQQWSSNPLTFGAG
TKLELKRADAAPTVSIFPPSSEQLTSGGASVVCFLNNFYPKDINVKWKID
GSERQNGVLNSWTDQDSKDSTYSMSSTLTLTKDEYERHNSYTCEATHKTS
TSPIVKSFNRNEC

In some embodiments, the anti-Langerin antibody comprises: a heavy chain wherein the variable domain has the the amino acid sequence set forth as SEQ ID NO:41, and a light chain wherein the variable domain has a sequence set forth as SEQ ID NO:42.

In some embodiments, the anti-Langerin antibody comprises: a heavy chain wherein the variable domain has the the amino acid sequence set forth as SEQ ID NO:43, and a light chain wherein the variable domain has a sequence set forth as SEQ ID NO:44.

In some embodiments, the anti-Langerin antibody comprises: a heavy chain having the the amino acid sequence set forth as SEQ ID NO:45, and a light chain having a sequence set forth as SEQ ID NO:46.

The antibodies of the invention may be produced by any technique known per se in the art, such as, without limitation, any chemical, biological, genetic or enzymatic technique, either alone or in combination. Knowing the amino acid sequence of the desired sequence, one skilled in the art can readily produce said polypeptides, by standard techniques for production of polypeptides. For instance, the antibodies of the invention can be synthesized by recombinant DNA techniques as is now well-known in the art. For example, these fragments can be obtained as DNA expression products after incorporation of DNA sequences encoding the desired (poly) peptide into expression vectors and introduction of such vectors into suitable eukaryotic or prokaryotic hosts that will express the desired polypeptide, from which they can be later isolated using well-known techniques.

In some embodiments, the amino acid sequence herein described comprise one or more sequences originating from the restriction cloning site(s) present in the polynucleotide encoding for said amino acid sequence. Typically, said sequences may consist of 2 amino acid residues and typically include AP, AS, AR, PR, SA, TR, and TS sequences.

In some embodiments, the amino acid sequences herein described the sequence of a signal peptide. As used herein, the term “signal peptide” has its general meaning in the art and refers to a pre-peptide which is present as an N-terminal peptide on a precursor form of a protein. The function of the signal peptide is to facilitate translocation of the expressed polypeptide to which it is attached into the endoplasmic reticulum. The signal peptide is normally cleaved off in the course of this process. The signal peptide may be heterologous or homologous to the organism used to produce the polypeptide.

In some embodiments, the antibody of the present invention comprises:
a heavy chain having at least 80% of identity with the amino acid sequence as set forth in SEQ ID NO:47, and
a light chain having at least 80% of identity with amino acid sequence as set forth in SEQ ID NO:48.

SEQ ID NO: 47
MGWSLILLFLVAVATRVHSEVQLVESGGGLVQPGGSLKLSCATSGFTFSDYYMYWVRQAPGKGLEWVAY
INSGGGSTYYPDTVKGRFTISRDNAKNTLYLQMNSLRAEDTAVYYCARRGLPFHAMDYWGQGTLVTVSS
ASTKGPSVFPLAPCSRSTSESTAALGCLVKDYFPEPVTVSWNSGALTSGVHTFPAVLQSSGLYSLSSVV
TVPSSSLGTKTYTCNVDHKPSNTKVDKRVESKYGPPCPPCPAPEFEGGPSVFLFPPKPKDTLMISRTPE
VTCVVVDVSQEDPEVQFNWYVDGVEVHNAKTKPREEQFNSTYRVVSVLTVLHQDWLNGKEYKCKVSNKG
LPSSIEKTISKAKGQPREPQVYTLPPSQEEMTKNQVSLTCLVKGFYPSDIAVEWESNGQPENNYKTTPP
VLDSDGSFFLYSRLTVDKSRWQEGNVESCSVMHEALHNHYTQKSLSLSLGKASQTPTNTISVTPTNNST
PTNNSNPKPNPASQNYTRSTDNQAMIKDALQSIQQQIKGLADKIGTEIGPKVSLIDTSSTITIPANIGL
LGSKISQSTASINENVNEKCKFTLPPLKIHECNISCPNPLPFREYKPQTEGVSNLVGLPNNICLQKTSN
QILKPKLISYTLPVVGQSGTCITDPLLAMDEGYFAYSHLEKIGSCSRGVSKQRIIGVGEVLDRGDEVPS
LFMTNVWTPSNPNTVYHCSAVYNNEFYYVLCAVSVVGDPILNSTYWSGSLMMTRLAVKPKNNGESYNQH
QFALRNIEKGKYDKVMPYGPSGIKQGDTLYFPAVGFLVRTEFKYNDSNCPIAECQYSKPENCRLSMGIR
PNSHYILRSGLLKYNLSDEENSKIVFIEISDQRLSIGSPSKIYDSLGQPVFYQASFSWDTMIKEGDVQT
VNPLVVNWRDNTVISRPGQSQCPRENKCPEVCWEGVYNDAFLIDRINWISAGVELDSNQTAENPVFTVE
KDNEVLYRAQLASEDTNAQKTITNCFLLKNKIWCISLVEIYDTGDNVIRPKLFAVKIPEQCTLK
SEQ ID NO: 48
DIQMTQSPSSLSASVGDRVTITCSASQGISNYLNWYQQKPGKAVKLLIYYTSILHSGVPSRESGSGSGT
DYTLTISSLQPEDFATYYCQQFNKLPPTFGGGTKLEIKRTVAAPSVFIFPPSDEQLKSGTASVVCLLNN
FYPREAKVQWKVDNALQSGNSQESVTEQDSKDSTYSLSSTLTLSKADYEKHKVYACEVTHQGLSSPVTK
SENRGEC

In some embodiments, the antibody of the present invention comprises:
a heavy chain having at least 80% of identity with the amino acid sequence as set forth in SEQ ID NO:47, and
a light chain having at least 80% of identity with amino acid sequence as set forth in SEQ ID NO:49.

SEQ ID NO: 49
DIQMTQSPSSLSASVGDRVTITCSASQGISNYLNWYQQKPGKAVKLLIYY
TSILHSGVPSRESGSGSGTDYTLTISSLQPEDFATYYCQQFNKLPPTFGG
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGECASKYKIKSNPLTKDIVIKMIPNVSNMSQCTGSVMEN
YKTRLNGILTPI

[symbol]

[symbol]

### Polynucleotides, Vectors and Host Cells of the Present Invention:

A further object of the invention relates to a polynucleotide that encodes for a heavy chain and/or light chain of the antibody of the present invention.

Typically, said polynucleotide is a DNA or RNA molecule, which may be included in any suitable vector, such as a plasmid, cosmid, episome, artificial chromosome, phage or a viral vector.

So, a further object of the invention relates to a vector comprising a polynucleotide of the present invention.

Such vectors may comprise regulatory elements, such as a promoter, enhancer, terminator and the like, to cause or direct expression of said antibody upon administration to a subject. Examples of promoters and enhancers used in the expression vector for animal cell include early promoter and enhancer of SV40, LTR promoter and enhancer of Moloney mouse leukemia virus, promoter and enhancer of immunoglobulin H chain and the like. Any expression vector for animal cell can be used, so long as a gene encoding the human antibody C region can be inserted and expressed. Examples of suitable vectors include pAGE107, pAGE103, pHSG274, pKCR, pSG1 beta d2-4 and the like. Other examples of plasmids include replicating plasmids comprising an origin of replication, or integrative plasmids, such as for instance pUC, pcDNA, pBR, and the like. Other examples of viral vector include adenoviral, retroviral, herpes virus and AAV vectors. Such recombinant viruses may be produced by techniques known in the art, such as by transfecting packaging cells or by transient transfection with helper plasmids or viruses. Typical examples of virus packaging cells include PA317 cells, PsiCRIP cells, GPenv+ cells, 293 cells, etc. Detailed protocols for producing such replication-defective recombinant viruses may be found for instance in WO 95/14785, WO 96/22378, U.S. Pat. Nos. 5,882,877, 6,013,516, 4,861,719, 5,278,056 and WO 94/19478.

A further object of the present invention relates to a host cell which has been transfected, infected or transformed by a polynucleotide and/or a vector according to the invention.

The polynucleotides of the invention may be used to produce an antibody of the present invention in a suitable expression system. Common expression systems include E. coli host cells and plasmid vectors, insect host cells and Baculovirus vectors, and mammalian host cells and vectors. Other examples of host cells include, without limitation, prokaryotic cells (such as bacteria) and eukaryotic cells (such as yeast cells, mammalian cells, insect cells, plant cells, etc.). Specific examples include E. coli, Kluyveromyces or Saccharomyces yeasts. Mammalian host cells include Chinese Hamster Ovary (CHO cells) including dhfr− CHO cells (described in Urlaub and Chasm, 1980) used with a DHFR selectable marker, CHOK1 dhfr+ cell lines, NSO myeloma cells, COS cells and SP2 cells, for example GS CHO cell lines together with GS Xceed™ gene expression system (Lonza), or HEK cells.

The present invention also relates to a method of producing a recombinant host cell expressing the antibody according to the invention, said method comprising the steps of: (i) introducing in vitro or ex vivo a recombinant polynucleotide or a vector as described above into a competent host cell, (ii) culturing in vitro or ex vivo the recombinant host cell obtained and (iii), optionally, selecting the cells which express and/or secrete said antibody.

Such recombinant host cells can be used for the production of antibodies of the present invention.

The host cell as disclosed herein are thus particularly suitable for producing the antibody of the present invention. Indeed, when recombinant expression are introduced into mammalian host cells, the polypeptides are produced by culturing the host cells for a period of time sufficient for expression of the antibody in the host cells and, optionally, secretion of the antibody into the culture medium in which the host cells are grown. The antibodies can be recovered and purified for example from the culture medium after their secretion using standard protein purification methods.

### Pharmaceutical and Vaccine Compositions:

The antibodies as described herein may be administered as part of one or more pharmaceutical compositions. Except insofar as any conventional carrier medium is incompatible with the antibodies of the present invention, such as by producing any undesirable biological effect or otherwise interacting in a deleterious manner with any other component(s) of the pharmaceutical composition, its use is contemplated to be within the scope of this invention. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatine; talc; excipients such as cocoa butter and suppository waxes; oils such as peanut oil, cottonseed oil; safflower oil, sesame oil; olive oil; corn oil and soybean oil; glycols; such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The antibodies as described herein are particularly suitable for preparing vaccine composition. Thus a further object of the present invention relates to a vaccine composition comprising an antibody of the present invention.

In some embodiments, the vaccine composition of the present invention comprises an adjuvant. In some embodiments, the adjuvant is alum. In some embodiments, the adjuvant is Incomplete Freund's adjuvant (IFA) or other oil based adjuvant that is present between 30-70%, preferably between 40-60%, more preferably between 45-55% proportion weight by weight (w/w). In some embodiments, the adjuvant is Polyinosinic-polycytidylic acid (poly (I:C)) or Polyinosinic-Polycytidylic acid-poly-L-lysine carboxymethylcellulose (Poly-ICLC). In some embodiments, the vaccine composition of the present invention comprises at least one Toll-Like Receptor (TLR) agonist which is selected from the group consisting of TLR1, TLR2, TLR3, TLR4, TLR5, TLR6, TLR7, and TLR8 agonists.

### Therapeutic Methods:

The antibodies as well as the pharmaceutical or vaccine compositions as herein described are particularly suitable for inducing an immune response against Nipah virus and thus can be used for vaccine purposes.

Therefore, a further object of the present invention relates to a method for vaccinating a subject in need thereof against Nipah virus comprising administering a therapeutically effective amount of the antibody of the present invention.

In some embodiments, the antibodies as well as the pharmaceutical or vaccine compositions as herein described are particularly suitable for the treatment of Nipah virus infection.

In some embodiments, the subject can be human or any other animal (e.g., birds and mammals) susceptible to Nipah virus infection (e.g., domestic animals such as cats and dogs; livestock and farm animals such as horses, cows, pigs, chickens, etc.). Typically said subject is a mammal including a non-primate (e.g., a camel, donkey, zebra, cow, pig, horse, goat, sheep, cat, dog, rat, and mouse) and a primate (e.g., a monkey, chimpanzee, and a human). In some embodiments, the subject is a non-human animal. In some embodiments, the subject is a farm animal or pet. In some embodiments, the subject is a human. In some embodiments, the subject is a human infant. In some embodiments, the subject is a human child. In some embodiments, the subject is a human adult. In some embodiments, the subject is an elderly human. In some embodiments, the subject is a premature human infant.

In some embodiments, the subject can be symptomatic or asymptomatic.

Typically, the active ingredient of the present invention (i.e., the antibodies and the pharmaceutical or vaccine compositions as herein described) is administered to the subject at a therapeutically effective amount. It will be understood that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; the activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. In particular, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, in particular from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

The antibodies and the pharmaceutical or vaccine compositions as herein described may be administered to the subject by any route of administration and in particular by oral, nasal, rectal, topical, buccal (e.g., sub-lingual), parenteral (e.g., subcutaneous, intramuscular, intradermal, or intravenous) and transdermal administration, although the most suitable route in any given case will depend on the nature and severity of the condition being treated and on the nature of the particular active agent which is being used.

The invention will be further illustrated by the following figures and examples. However, these examples and figures should not be interpreted in any way as limiting the scope of the present invention.

### FIGURES

FIG. 1. Epitope mapping of NiV G antigen associated to the CD40 mAb. Within the genome of NiV, ORF encoding the G, F and N proteins are highlighted in blue, orange and red respectively. Full-length aa sequences were screened for predicted HLA-I (NetMHC 4.0) and -II (NetMHCII 2.3) and linear B cell epitopes (BepiPred 2.0). For HLA-I and —II, colors intensity is representative of the density of predicted epitopes, with at least of coverage of 30% of the worldwide HLA. (Top) Ectodomain of the G protein associated to Gen-1 and Gen-2 vaccines.

FIG. 2. Epitope mapping of NiV F and N antigens associated to the CD40 mAb. F and N downselected domains associated to Gen-2 vaccine are represented.

FIG. 3. Schematic representation of Generation 1 (Gen-1) and Generation 2 (Gen-2) vaccines.

FIG. 4 Gen-1, Gen-2 and IgG4 control vaccine SDS PAGE profiles in non-reduced (NR) versus reduced (R) conditions.

FIG. 5: The G antigen fused to Gen-1 vaccine was confirmed by Western-Blot, using sera from Niv(G)-immunized mice and testing the in-house produced G protein as positive control. Molecular weight (kDa) markers are shown in the left lane. Arrows indicate the heavy chain (HC) and light chain (LC) of the vaccine constructions at the expected size, respectively.

FIG. 6: Size exclusion chromatography (SEC) of Gen-1 and Gen-2 vaccines with standard proteins size markers. Recombinant mAb are indicated by arrows.

FIG. 7: Binding assay of CD40.NiV vaccines to mouse cells expressing the human CD40 receptor. Mouse splenocytes from hCD40^{My/Hu} Tg mice were incubated with 10 nM of CD40.Niv Gen-1 mAbs and subsequently labeled for T-, B and DC cell markers, respectively. Untreated and splenocytes treated with the non-targeting IgG4.Niv were used as negative controls.

FIG. 8: Binding assay of CD40.NiV vaccines to AGM PBMCs. Same method as FIG. 7, using PBMCs from naïve AGM. Two in-house produced anti-CD40 mAbs that were not associated with the G protein (clone VH3 and 11B6) were used as positive controls. Binding of the vaccine was demonstrated on B cells which express CD40, and to a less extend to monocytes/macrophages population.

FIG. 9: Gen-1 and -2 CD40.NiV vaccines induced humoral immune responses in hCD40^{Hy/Mu} transgenic mice. G-specific IgG titers was determined by ELISA at day 0, 21 and 28 post-immunization of mice immunized twice with 10 ug of CD40.NiV (Gen-1), Gen-2, or molar equivalent of the Niv(G) protein alone, with (plain circles) and without Poly-ICLC (open circles). Medians of AUC [Min−Max] are represented and compared to control animals (Poly-ICLC alone).

FIG. 10: Neutralization activity of mice sera after vaccination detected by Luminex-based pseudo-neutralization assay at day 0, 21 and 28 post-immunization. Statistical analysis are same as in FIG. 9

FIG. 11: in vitro neutralization assay was performed on NiV-B infected cells using serial-diluted sera collected at d28 post-immunization. IC₅₀ was calculated and reported as titers. Statistical analysis are same as in FIG. 9.

FIG. 12: Niv(G)-specific B cells in dLN were stained using biotinylated NiV(G) proteins. (left) Representative dot plots. Niv(G)-specific B cells are considered double positive for the two anti-Biotin mAbs. (right) Percentage (%) of GC B-cells (FAS+GL-7+) and Percentage Niv(G)-specific B cells among this population. Animals were immunized with 30 ug of CD40.NiV (Gen-2) with Poly-ICLC and were compared to the Poly-ICLC negative group. Mann-Whitney unpaired t-test, **, p<0.01; ***, p<0.0001.

FIG. 13: Gen-1 and -2 CD40.NiV vaccines induced cellular immune responses in hCD40^{Hy/Mu} transgenic mice. IFN-g T cells responses to the G, F and N overlapping pools of peptides were assessed by ELISpot, one week post boost. (left) Specific IFN-g responses to the G antigen of mice immunized by Gen-1 and Gen-2, respectively. Number of spots are reported per million of splenocytes (right) Total specific IFN-g responses to the three antigens of Gen-2, in mice immunized with 10 and 30 ug of vaccine, respectively. (Bottom right) Percentages of G, F and N-specific responses, at 10 and 30 ug of vaccine, respectively. Plain circle, with Poly-ICLC, open circles, without Poly-ICLC.

FIG. 14: Immune responses of AGM vaccinated with CD40.Niv Gen-2. Schematic representation of the study design. Diamonds are representative of sampling. Nine AGM were immunized with CD40.NiV(Gen-2) with Poly-ICLC with 3-week prime-boost interval. Niv(G)-specific IgG (left) and IgA (right) titers, in the Poly-ICLC animal group (n=3, left) vs the vaccinated animal group (n=9, right), at 0, 10, 21, 35 and 56 days post-prime, respectively. Individual Ig titers (log(1/EC₅₀)) are represented, and whiskers indicate medians [Min−Max] for each group and time-point, respectively. Prime and boost are indicated by arrows. Dotted line, threshold of Ig titer detection.

FIG. 15: As in FIG. 10, neutralization activity of AGM sera was measured by indirect inhibition assay

FIG. 16: In vitro neutralization of NiV-B by sera from vaccinated AGM.

FIG. 17: As in FIG. 13, total specific IFN-γ responses to the G, F and N antigens were measured in AGM circulating PBMCs collected at day 21 and 35 post-immunization. Number of spots are reported per million of PBMC. Pointed line represents threshold for positivity defined by the mean responses (+3SD) of the non-vaccinated AGM group. (right) Percentage (%) of specific T cell response to each vaccine antigen (mean±SD).

FIG. 18: In vitro NiV-B neutralization activity of AGM sera measured post immunization and post-challenge (day 80 pi): poly-ICLC group (n=3, black circle); CD40.NiV (Gen-2) with poly-ICLC (n=9, white circle); thin black line, modeling of the decrease in neutralizing Abs after the peak of the response (day 35).

FIG. 19: Protection assay in AGM. Survival curve. Nine AGM were immunized twice with CD40.NiV (Gen-2) and challenged with 10² Pfu NiV-B (intratracheal route). Height naïve animals were used as controls. Gehan-Breslow-Wilcoxon test, ****, p<0.0001.

FIG. 20: Clinical scores and temperatures of naïve (black circle) and vaccinated AGMs (white circle). Dotted line score threshold for ethical considerations (left panel); dotted line: median temperature at day 0 (right panel).

FIG. 21: Viral dissemination post-challenge. (A) (left) Amount of viral NiV RNA quantified by RT-PCR (N gene) in PBLs. Values were normalized against those of the GADPH housekeeping gene. Genomic RNA was measured by RT-qPCR in nasal (middle) and pharyngeal (right) swabs. Mean values (±SEM) are presented for the non-vaccinated (black circle, n=7) versus CD40.NiV (Gen-2) vaccinated (white circle, n=9) groups. (B) Same as in A for fluids collected at necropsy.

### EXAMPLE

### Methods

### Molecular Cloning and Production:

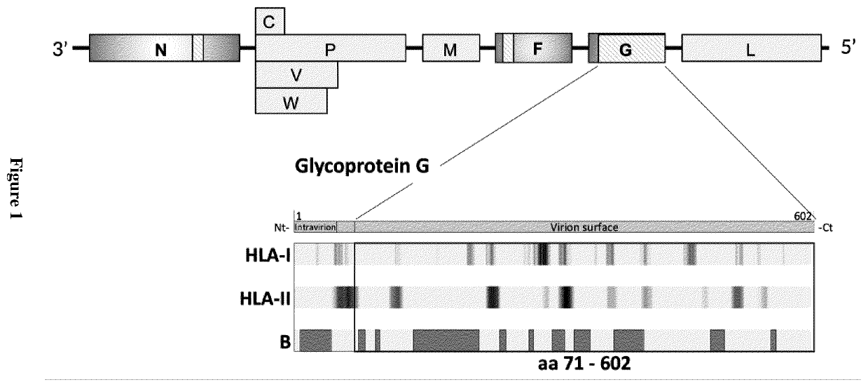

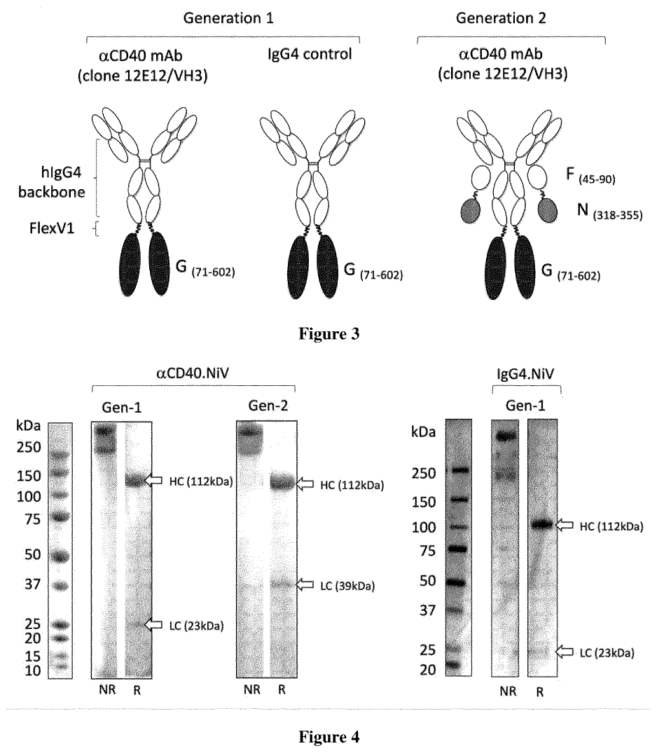

Generation-1: The ectodomain of the NiV-B G protein (FIG. 1, The Bangladesh strain (NiVB) (GenBank: AY988601.1) (Harcourt B H et al., Emerging Infect. Dis. 2005) ectodomain highlighted) was cloned into the UCOE vector, at the C-terminal end of the Heavy-chain of the anti-CD40 mAb (clone 12E12/VH3) with a flexible linker (see FIG. 3).

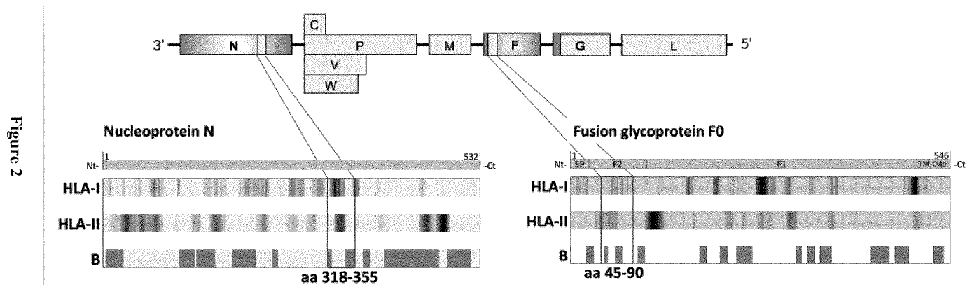

Generation-2: AA sequence of the F and N proteins (strain Bangladesh) were screened for the density of predicted MHC-I and -II epitopes and the diversity of HLA using online software (NetMHC) (Figure. 2). Sequences were further breakdown by analyzing the presence of predicted linear B cell epitopes, and by screening non-hydrophobic stable protein domains. One peptide in F and one in N were associated with linker domains to the C-terminal end of the anti-CD40 light chain (clone 12E12/VH3) (see FIG. 3 right).

### Production in CHO Cell Line:

CHO cells were transfected by the plasmids expressing H- and L-chains (see FIG. 3). Culture supernatant were collected, protein A captured and purified on FPLC. Purified vaccine batches were controlled for their endotoxin levels (<0.5 ng/mg of protein).

### Biochemical Quality Controls:

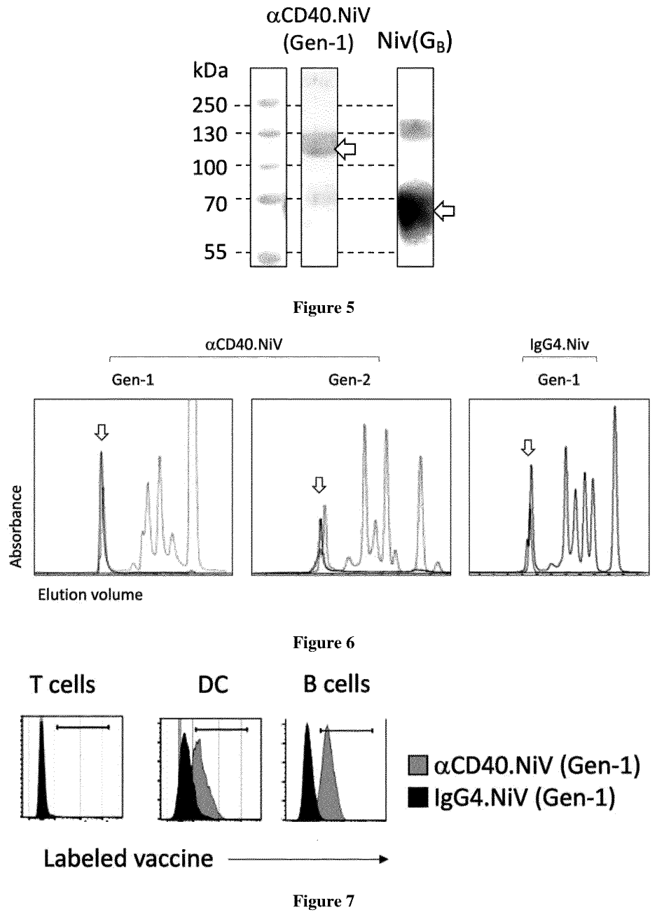

Quality of vaccine batches was analyzed by 1) SDS-PAGE analysis (under reduced and non-reduced conditions) followed by a Coomassie Blue staining (FIG. 4), and ii) a SEC analysis using standard proteins (FIG. 6). Gen1 vaccine was assessed by western blot (FIG. 5), using sera from C57BL/6 mice immunized by the in-house produced G protein (+CpG). The H-chain fused with the G protein was revealed by the mouse sera.

### Binding Assay:

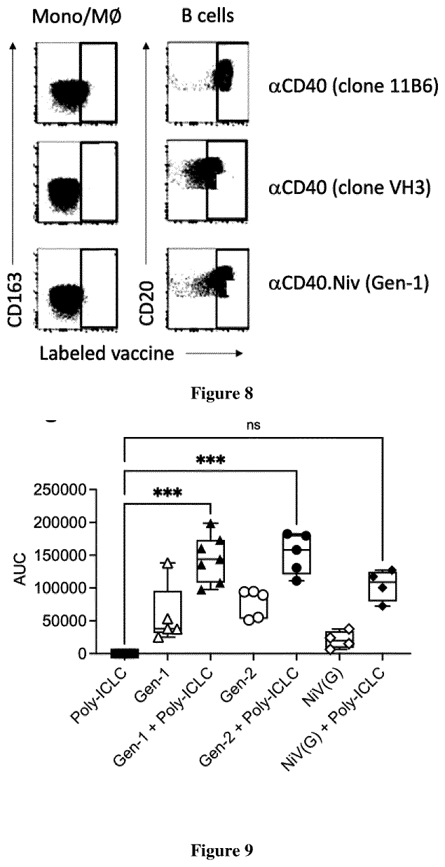

Gen-1 antibodies and same 12E12/VH3 clone without any antigen were marked by a fluorochrome. The clone 11B6 was also tested in parallel as positive control. Mouse splenocytes from hCD40 Tg mice (FIG. 7) and PBMCs from AGM (FIG. 8) were incubated with fluorescent vaccine in vitro. Cells were phenotyped and analyzed by FACS.

Immunological Responses in hCD40 Tg Mice:

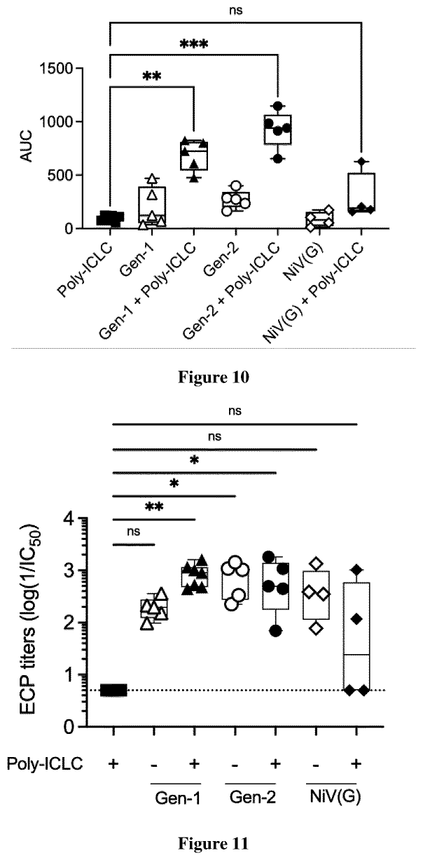

hCD40^{Hy/Mu} Tg mice (CO-1704 hTnfrsf5-OST5(CD40) Knock-in HOM) were immunized subcutaneously twice with either CD40.NiV(G) (Gen-1 or -2) or the Niv(G) protein alone with or without any adjuvant (see Table 1). Sera were collected and week 0, 3 and 4. Splenocytes were collected at week 4. Sera were tested by ELISA for their amount of G-specific IgG. Serial dilution were assessed to determine antibody titers (Figure-9). Neutralizing activity of serum Ab was measured by in house Luminex assay (FIG. 10) or in vitro NiV-B neutralization assay (FIG. 11). Serial dilution were performed to determine neutralization effect of the sera on NIV infection. Formation of Germinal center B cells and G-specific B cells within this population was estimated by FACS (Figure-12).

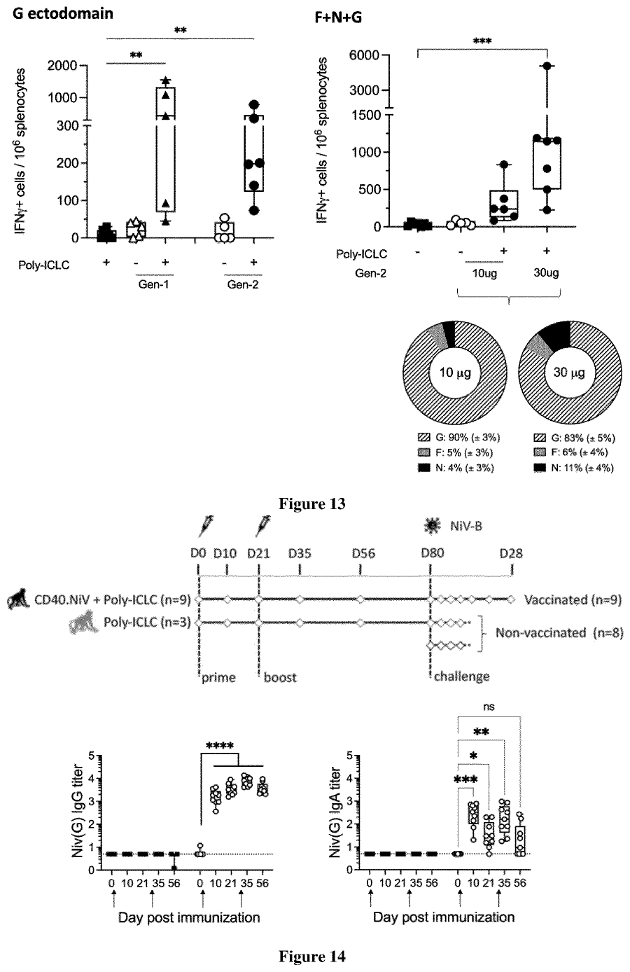

Splenocytes were stimulated in vitro with either a pool of overlapping peptides (OVLP at 1 μg/mL; FIG. 13) encompassing the downselected domains from the NiV G, F and N proteins contained within the vaccine (FIGS. 1 and 2). IFN-g production was assessed by ELISpot.

### Immunological Responses in AGM:

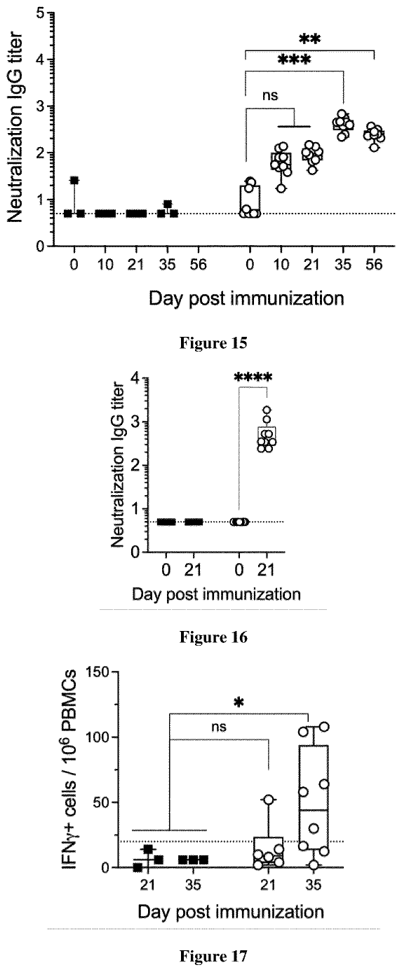

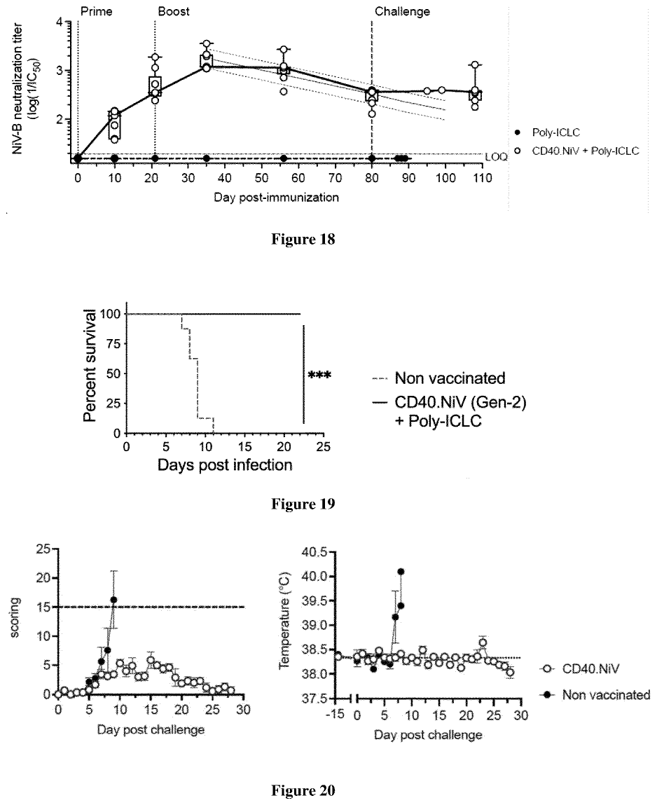

Nine AGM were vaccinated twice with CD40.NiV(Gen-2) vaccine with 3-week intervals. Three naïve animals were used as controls. Blood draws were regularly collected to measure by ELISA G-specific IgG and IgA (Figure-14) in sera. Neutralizing activity of sera was determined as in mouse studies (FIGS. 15, 16, and 18). IFNg-ELISPOT was performed on PBMCs collected at day 21 and 35 post-immunization (FIG. 17).

### Challenge Assay:

Vaccinated animals (n=9) versus naïve AGM (n=8) were challenged intratracheally with 10² pfu of Niv-B. Survival was observed over three weeks (FIG. 19).

### Clinical and Hematobiochemical Follow-Up:

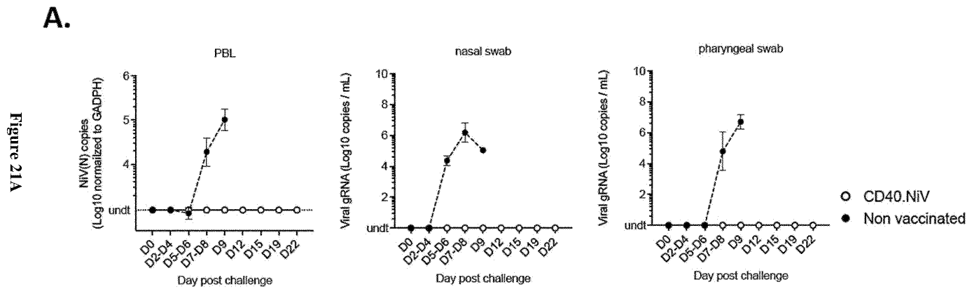

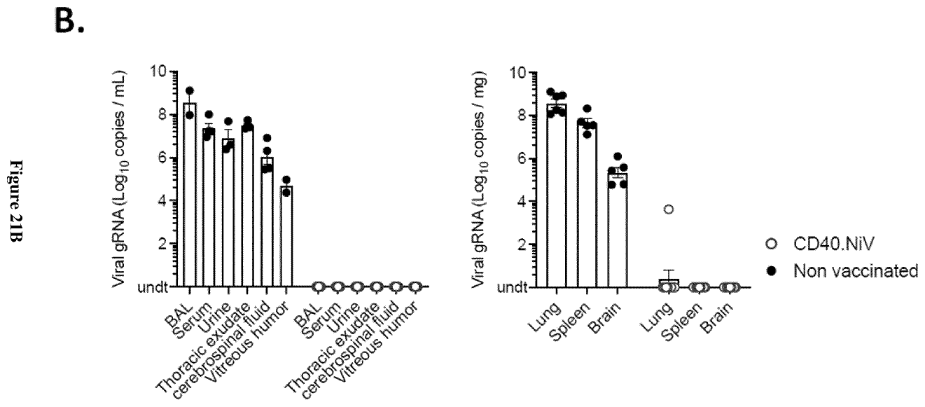

Clinical exams were performed everyday post-challenge and the following parameters were scored: temperature, weight, dehydration, breath, reactivity, feces exams and neurological symptoms. Animals were euthanized when the total score reached >15 for ethical reasons (FIG. 20).

### Virology

RNA Extraction from Infected AGM PBL:

Blood samples from infected AGMs collected in EDTA tubes were diluted in Pharmalyse buffer, vortexed, and incubated in the dark for 15 min. They were then centrifuged at 200×g for 5 min and the pellets washed in 2 mL PBS 1% FBS, 2 mM EDTA. Finally, pellets were resuspended in 600 μL RLT lysis buffer and purified following the manufacturer's instructions (Macherey Nagel). Samples were stored at −80° C. till use.

### RT-qPCR:

Viral RNA was extracted using a Qiamp Viral RNA Kit (Qiagen) for serum, swabs, urine, BAL, vitreous humor, and thoracic exudate samples and a Nucleospin Kit (Macherey Nagel) for PBMCs and organs. Viral load was evaluated by one-step RT-qPCR using NiV-N-specific primers and GAPDH primers, if necessary (FIG. 21).

### Results

### In Silico Down-Selection of NiV Predictive Immunogenic Peptides and CD40.NiV Vaccine Design

Two CD40.NiV vaccine candidates have been successfully produced. While Gen-1 and -2 contains the gold-standard Niv G (glycoprotein) surface antigen, Gen-2 vaccine is also associated with in silico down selected peptides from the F and N proteins, and that are predicted to be enriched in T- and B-cells epitopes (FIGS. 1 to 8). The NiV surface glycoprotein (G) is a gold-standard antigen for inducing protective humoral responses. Other cellular effectors, such as helper and effector T-cells, may also participate in host defense. We screened the NiV G ectodomain (ECD) to identify vaccine epitopes using NetMHC 4.0 and NetMHCII 2.3 software, which predict T-cell epitopes that bind to a large panel of class-I and -II HLA molecules, respectively. Linear B-cell epitopes were predicted using BepiPred 2.0. The NiV G ECD vaccine region was predicted to contain 3522 and 15 T- and B-cell epitopes, respectively. Due to their conservation between NiV strains, we further identified vaccine epitopes from the fusion (F) and nucleocapsid (N) proteins. Regions with strong binder epitopes and the highest HLA coverage were highlighted, as well as linear B-cell epitopes. The NiV F (aa 45-90) and NiV N (aa 318-355) down-selected peptides contain 356 and 266 predicted T-cell epitopes and three and linear B-cell epitopes, respectively. Globally, these amino acid sequences were screened for homology with other Henipaviruses, obtaining 100% homology between different Nipah strains for the F and N peptides and more than 98% for NiV G ECD. (FIG. 1 to 8) We next engineered vectors expressing NiV-B G ECD fused to the C-termini of the Heavy Chains (HCs) of the anti-human CD40 humanized 12E12 IgG4 monoclonal antibody, with additional selected peptides of NiV F and NiV N fused to the C-termini of the Light chains (LC) (named CD40.NiV) (FIG. 3). Vaccines were produced in CHO cells and controlled for their quality. We confirm, here, binding of the CD40.NiV vaccine to the human CD40 and AGM CD40 receptor in vitro using splenocytes from mice transgenic for the human CD40 receptor (CD40Hy/Mu transgenic mice; hCD40Tg) and PBMCs from AGM, respectively (FIG. 7-8)

CD40.NiV Induces Specific T- and B-Cell Responses in hCD40Tg Mice

We first evaluated the immunogenicity in hCD40^{my/Hu} Tg mice. hCD40Tg mice were immunized with two subcutaneous (SC) injections of CD40.NiV (Gen-1 or Gen-2) vaccine (10 μg) or an equivalent amount of NiV G protein, both with poly-ICLC (50 μg) on days 0 and 21. Antibody-mediated immune responses were first evaluated by Luminex assay. Our results showed a significant G-specific IgG titer with both vaccines (FIG. 9). Three weeks post-prime, mice immunized with the CD40-targeted NiV G protein showed significantly higher anti-NiV G IgG levels (P<0.01), highlighting the benefit conferred by the DC-targeting system. This antibody level was markedly improved i) when the G protein was targeted to APCs, and ii) when mice were immunized with adjuvant. We next compared the avidity of NiV G-specific IgG post-boost using a multiplex immunoassay approach. Strikingly, the avidity index was significantly improved when NiV G was targeted to the CD40 receptor (P<0.05), suggesting an advantage of such targeting for inducing B-cell affinity maturation (data not shown). Noteworthy, we set-up an in-house Luminex-based assay to assess the inhibition of the binding of G to the Ephrin B2 receptor by the IgG circulating in the sera. Our results revealed a strong capacity of immunized sera to pseudo-neutralize this protein G/receptor interaction (FIG. 10). Strikingly, the neutralization capacity of vaccine-induced IgG was confirmed by a neutralization assay performed at the BSL-4 (FIG. 11).

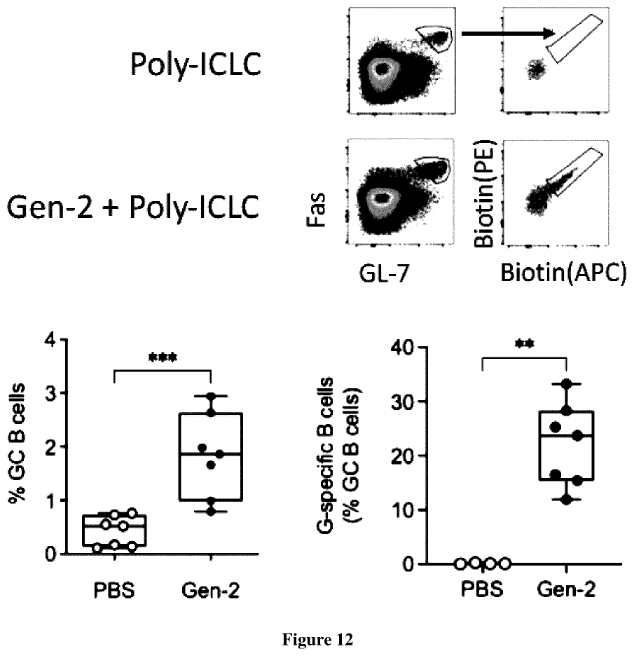

G-specific B cells were detected by FACS within the germinal center B cells populations of immunized mice (FIG. 12). We further confirmed the induced B-cell responses in CD40.NiV (+poly-ICLC) vaccinated mice by detecting germinal center (GC) B-cells within the draining lymph nodes (dLN) (P<0.001 as compared to the Poly-ICLC control group) (FIG. 12). Furthermore, FACS-staining showed a significant NiV G-specific GC B-cell population in the dLN of vaccinated animals (P<0.01). To demonstrate the specific T-cell responses against the various antigens from CD40.NiV, we performed an IFN-g ELISpot assay on splenocytes using pools of overlapping peptides (FIG. 13). We detected dose-dependent IFN-g producing cells specific for NiV G ECD, as well as NiV F and N peptides. Last, we demonstrated by IFN-g ELISpot performed on mouse splenocytes that Gen-2 vaccine induced cellular responses specific to the G, F and N peptides compromised within the vaccine, respectively (FIG. 13). Overall, these results demonstrate the immunogenicity of the CD40.NiV vaccine candidate.

### The CD40.NiV Vaccine Induces Early and Robust Humoral and T-Cell Responses in AGMs

We then assessed cellular and humoral responses of the Gen-2 vaccine in the African Green Monkey (AGM) model, which is the most relevant model to test the infection by the Bangladesh NiV strain. Twelve animals have been imported and housed at the Bioprim animal center (Toulouse). Two groups of animals have been immunized in a prime-boost homologous regimen, as follow: 3 AGM with Poly-ICLC alone versus 9 AGM with 200 ug of Gen-2 vaccine+Poly-ICLC. A serial collection of blood samples have been performed as follow: (i) sera for G-specific Ig titer and neutralization, and (ii) PBMCs for IFN-g ELISpot (FIG. 14). We demonstrated that our vaccine candidate induced a strong and long-lasting humoral response, with circulating IgG neutralizing the virus in vitro (assessed in BSL-4) (FIGS. 14 to 16). All vaccinated animals showed specific and significant IgG and IgA titers 10 days post-prime (FIG. 14). IgG titers increased over time and then remained the same until day 56, whereas serum IgA levels dropped. Vaccination elicited a neutralization potential of NiV G-specific IgG by day 10 post-prime, reaching significance two weeks post-boost (mean neutralization titer 3.2 (±0.2), P<0.001), and remained high until day 56 (FIGS. 15, 16 and 18). We estimated a significant average decrease of 0.016 logs neutralization titer per day, leading to a prediction of the maintenance of a neutralization titer above the detectable threshold (mean log titer 2.2 (±0.1)) up to 100 days following the boost. The neutralizing capacity of specific antibodies was confirmed post-prime and post-boost using the Luminex-based surrogate inhibition assay (P<0.001) (data not shown). We assessed cross neutralization to NiV-B, -M (Malaysia) and -C (Cambodia), and HeV using the sera of five vaccinated AGMs by in vitro infection of VeroE6 cells with recombinant (F/G)VSV particles (data not shown). Two weeks post-boost, all animals exhibited comparable cross-reactive humoral responses to NiV (F/G) proteins, as well as positive cross-reactive responses to Hendra virus (data not shown). IFN-g ELISpot assays were conducted on PBMCs collected on day 21 (before the boost) and day 35 post-prime, using pools of peptides for each vaccine antigen (FIG. 17). The overall responses were significant two weeks post-boost (P<0.05) and positive in 5/8 tested animals. Our results highlighted a polyclonal IFN-g T cells responses to all vaccine antigens (FIG. 17).

Overall, CD40.NiV (Gen-2) induced cellular responses associated with strong, rapid and durable humoral responses in an AGM model, with high titers of neutralizing antibodies.

### Clinical Outcomes and Survival for Challenged Animals

Then we tested vaccine-induced protection against Niv infection in African green monkeys (AGM). Control and vaccinated AGM have been transferred to the BSL-4 where they have been challenged with Niv (10² pfu intratracheal, Bangladesh strain). The dose for the challenge was selected based on previous experiments that included five non-vaccinated animals infected under the same conditions. Strikingly, we observed a complete protection of vaccinated animals (at Day 22 post-challenge, n=8), whereas all non-vaccinated animals died between D7 and D11 (n=8) (FIG. 19). From a clinical standpoint, only mild and transient clinical signs were evident in the vaccinated animals, with a mean clinical score remaining below 6, mostly attributed to a lack of reactivity (FIG. 20). The control group showed a high grade clinical score, with apathy, tachypnea, dyspnea, and gastrointestinal symptoms, along with fever (above 38.9° C. in all animals). The animals showed various hematological and serum biochemistry changes during the challenge phase (data not shown). Enzyme activity levels (aspartate aminotransferase, AST; creatine kinase, CK) reflecting hepatic disorders were above normal values during the critical disease period for controls but not the vaccinated animals. We also observed perturbations of the white and red blood counts in the controls, which exhibited lymphopenia and thrombocytopenia, whereas we observed no significant abnormalities in the vaccinees during the post-immunization and challenge phases. Necropsy studies revealed lesions and the pathophysiological process of the NiV-B infection in control animals (data not shown). Examination of the lung tissue showed interstitial pneumonia, edema, and vasculitis, with inflammatory cell infiltrates for all controls but rarely for the vaccinated animals (observed in 2 of 9 animals, AGM #S1134 and #O1376, data not shown).

Non-vaccinees showed follicular depletion in the spleen, whereas eight CD40.NiV-vaccinated AGMs showed follicular hyperplasia, suggesting that a strong adaptive immune response was induced post-challenge. Of note, we observed no relevant lesions in the frontal cortex of any animals. From a clinical point of view, CD40.NiV (Gen-2) appears to confer full protection against disease progression and death.

### Plasma and Tissue Viral Loads in Challenged AGMs

We assessed NiV viremia by qRT-PCR in PBLs from vaccinated and control animals at regular time-points and at termination of the study (euthanasia or 28 dpc) for organs, fluids, and swabs. All controls (n=3 receiving poly-ICLC plus n=4 naïve AGM) exhibited a high level of NiV-B in PBLs (ranging from 4.5-5.5 log 10 copies/mL) detectable from 7 to 8 dpc, whereas we detected no virus in vaccinated subjects until 22 dpc (FIG. 21A). Except for one animal (AGM #S1134), for which NiV-B transcripts were detected by RT-PCR in the lung, NiV-B replication in nasal and nasopharyngeal swabs, fluids, including broncho-alveolar lavages (BALs), sera, urine, and thoracic exudates, and organs (lung, spleen, and neurological tissue) was undetectable in immunized AGMs, whereas it was detected at high levels in controls (FIG. 21B). This strong antiviral effect was confirmed by the lack of expression of the NiV N protein in tissues of the lungs and spleen, as assessed by histo-immunofluorescence (data not shown). Viral syncytia were detected in the lungs of non-immunized animals. Of note, NiV-B infection of the brain could not be confirmed by histo-immunofluorescence. Overall, CD40.NiV (Gen-2) vaccination led to sterilizing immunity that limited viral propagation, as well as viral shedding, in AGMs.

### Immunological and Cytokine Features of Challenged Animals

We further characterized the post-challenge immune responses by immunological phenotyping of the cell populations from 0 to 22 dpc. The succumbing animals showed profound and significant defects in the lymphoid populations (CD20+B- and CD3+ T-cells) (data not shown). Among T cells, the percentage of CD8+ T-cells was markedly reduced during NiV infection (data not shown). These changes were transitional and not significant in the vaccinated group. Innate immunity was also affected by NiV-B infection, with the almost complete disappearance of circulating monocytes in succumbing animals, whereas the number of inflammatory, intermediate, and classical monocytes in the vaccinees remained stable after the critical period of infection (7 to 9 dpc) (data not shown). Overall, these data indicate that exposure to NiV challenge induces dramatic perturbations of innate and adaptive cellular immunity, which are attenuated and/or nonsignificant in CD40.NiV-immunized animals.

### Changes in Blood-Cell Gene Expression in Vaccinated AGM

To decipher early changes in gene expression associated with the vaccination, we performed RNA sequencing analysis of peripheral whole blood at DO (before prime) and one day post-prime (PP, D1), just before the boost (D21), and one day post-boost (PB, D22) in animals receiving adjuvanted CD40.NiV(Gen-2). Given the small number of animals in the adjuvant control group, we analyzed the differentially expressed genes (DEGs) at different time points relative to baseline in the vaccinated animals. Principal component analysis (PCA) showed changes in gene abundance PP and PB relative to baseline (DO before injection) (data not shown). In total, 773 DEGs were significantly modulated at D22 (one day post-boost). Interestingly, among these DEGs, 437 were noted at D1 and D22 (one day PP and PB). An additional 236 DEGs were common between day 1 PP and PB (data not shown). The most highly upregulated genes from DO to D22 included those that play a major role in antiviral innate and proinflammatory responses (e.g., ISG15, MX1, IFI44, CXCL10, and IL-27) and adaptive immunity (e.g., antigen uptake by SIGLEC-1, T-cell chemotaxis by CCL8, and CXCL11) (data not shown). Most of these transcripts were found to be upregulated PP and PB (data not shown). A panel of the most highly downregulated genes was also associated with a signature of a primary or secondary immune response to the vaccine (e.g., CD1c, CD79A, CCR6, and CXCR4). Globally, the most highly upregulated and downregulated genes in vaccinated animals were predominantly associated with pathways involved in immune system processes and the defense against viruses and other organisms (data not shown). The humoral immune response mediated by circulating immunoglobulin was specifically promoted PB (data not shown), highlighting a prominent antibody-mediated response after the second injection of CD40.NiV (Gen-2).

Overall, the antiviral defense module of genes was the most significantly upregulated on D22 (data not show). The detailed kinetics of the genes involved in this module showed rapid and significant activation of all PP and PB (data not shown). Ten transcripts, including interferon-inducible antiviral proteins (RSAD2 and RIG1), were significantly upregulated PB relative to PP. Other gene network modules were also highlighted PP and PB (e.g., response to virus, cytokine signaling, neutrophil degranulation, and regulation of leukocyte activation). Although we could not discriminate between effects of the vaccine and the adjuvant in the vaccinees, our transcriptomic analysis highlights a vaccine-induced gene signature.

### CONCLUSION

Here, we demonstrate the potency of an innovative DC-targeting vaccine candidate to prevent NiV-B infection in challenge experiments in an AGM model. More than three decades after discovering the immunological properties of DCs, we prove, for the first time, that targeting viral antigens to professional APCs can be efficiently used as a prophylactic means against a viral challenge at a lethal dose. The DC-targeting strategy allowed us to design a subunit construct containing immunogenic and cross-reactive epitopes from the F and N NiV proteins, in addition to the NiV G ECD, in contrast to most other NiV vaccine platforms (Bossart et al., 2012; Foster et al., 2022; Geisbert et al., 2021; 287 Loomis et al., 2020; Mohammed et al., 2020; Woolsey et al., 2023; Yoneda et al., 2013).

The CD40.NiV vaccine induced both IgG and IgA antibodies as early as 10 days post-prime in AGMs. We also show that neutralizing responses can be maintained, with a stable estimated mean log titer of approximately 2.2 (±0.1) 100 days after the peak of the Ab response.

Responses were showed to cross-neutralize multiple strains of NiV, but also HeV. Although detectable at a lower level, we found that the CD40.NiV vaccine elicited a T-cell response against the NiV G ECD and down-selected F and N peptides in two preclinical models. Transcriptomic analysis showed a common set of DEGs one day following prime and boost (D1 and D22), including those for antiviral innate (ISG15, MX1, IF144) and adaptive immunity. The differentially expressed genes in vaccinated animals were predominantly associated with pathways involved in immune system processes and the defense against viruses involving innate immunity at earlier time point (D1, PP) as described with other vaccine strategies (Hagan et al., 2022). Interestingly, the modulation of gene pathways associated with humoral responses was significant post-boost at day 22.

Overall, these results highlight a vaccine signature associated with the protective effect of the vaccine.

### REFERENCES

Throughout this application, various references describe the state of the art to which this invention pertains. The disclosures of these references are hereby incorporated by reference into the present disclosure.

1. An antibody that is directed against a surface antigen of an antigen presenting cell wherein a heavy chain and/or a light chain of the antibody is conjugated or fused to a polypeptide having at least 80% identity with the amino acid sequence that ranges from the amino acid residue (Q) at position 71 to the amino acid residue (T) at position 602 in an Niv(G)_{B} ectodomain having an amino acid sequence as set forth in SEQ ID NO: 1.

2. The antibody of claim 1 wherein the light chain of the antibody is conjugated or fused to the polypeptide having at least 80% of identity with the amino acid sequence that ranges from the amino acid residue (Q) at position 71 to the amino acid residue (T) at position 602 in SEQ ID NO:1.

3. The antibody of claim 1 wherein the heavy chain of the antibody is conjugated or fused to the polypeptide having at least 80% of identity with the amino acid sequence that ranges from the amino acid residue (Q) at position 71 to the amino acid residue (T) at position 602 in SEQ ID NO:1.

4. The antibody according to claim 1 wherein the heavy chain and/or the light chain of the antibody is conjugated or fused to the Niv(G)B ectodomain via its C-terminal.

5. The antibody according to claim 1, wherein the heavy chain and/or the light chain of the antibody is fused to the N-terminal of the Niv(G)B ectodomain.

6. The antibody according to claim 1, wherein the heavy chain and/or the light of the antibody is fused to the Niv(G)B ectodomain to form a fusion protein.

7. The antibody according to any one of claim 1 wherein the Niv(G)B ectodomain is fused either directly or via a linker to the heavy and/or the light chain.

8. The method of claim 7, wherein the linker is selected from the group consisting of SEQ ID NO:5 (FlexV1), SEQ ID NO:6 (f1), SEQ ID NO:7 (f2), SEQ ID NO:8 (f3), or SEQ ID NO:9 (f4).

9. The antibody according to claim 1, wherein the antibody comprises:
a heavy chain conjugated or fused to the polypeptide having at least 80% identity with the amino acid sequence that ranges from the amino acid residue (Q) at position 71 to the amino acid residue (T) at position 602 in SEQ ID NO: 1 and
a light chain is conjugated or fused to the polypeptide having at least 80% identity with the amino acid sequence that ranges from the amino acid residue (K) at position 45 to the amino acid residue (I) at position 90 SEQ ID NO: 2;
or
a heavy chain conjugated or fused to the polypeptide having at least 80% identity with the amino acid sequence that ranges from the amino acid residue (Q) at position 71 to the amino acid residue (T) at position 602 in SEQ ID NO: 1 and a light chain is conjugated or fused to the polypeptide having at least 80% of identity with the amino acid sequence that ranges from the amino acid residue (I) at position 318 to the amino acid residue (L) at position 355 in SEQ ID NO: 3,
or
a heavy chain conjugated or fused to the polypeptide having at least 80% identity with the amino acid sequence that ranges from the amino acid residue (Q) at position 71 to the amino acid residue (T) at position 602 in SEQ ID NO: 1 and
a light chain conjugated or fused to both i) the polypeptide having at least 80% identity with the amino acid sequence that ranges from the amino acid residue (K) at position 45 to the amino acid residue (I) at position 90 in SEQ ID NO: 2 and ii) the polypeptide having at least 80% of identity with the amino acid sequence that ranges from the amino acid residue (I) at position 318 to the amino acid residue (L) at position 355 in SEQ ID NO: 3;
or 12
a heavy chain conjugated or fused to a polypeptide having at least 80% identity with the amino acid sequence that ranges from the amino acid residue (Q) at position 71 to the amino acid residue (T) at position 602 in SEQ ID NO: 1 and,
a light chain conjugated or fused to i) a polypeptide having at least 80% of identity with the amino acid sequence that ranges from the amino acid residue (I) at position 318 to the amino acid residue (L) at position 355 in SEQ ID NO: 3 and ii) the polypeptide having at least 80% of identity with the amino acid sequence that ranges from the amino acid residue (K) at position 45 to the amino acid residue (I) at position 90 in SEQ ID NO: 2.

10. (canceled)

11. (canceled)

12. (canceled)

13. The antibody of claim 9, wherein the polypeptide having at least 80% identity with the amino acid sequence that ranges from the amino acid residue (I) at position 318 to the amino acid residue (L) at position 355 in SEQ ID NO: 3 is fused via a linker to the polypeptide having at least 80% of identity with the amino acid sequence that ranges from the amino acid residue (K) at position 45 to the amino acid residue (I) at position 90 in SEQ ID NO: 2.

14. The antibody of claim 13 wherein the linker consists of the amino acid sequence as set forth in SEQ ID NO: 10.

15. The antibody of claim 14 wherein the antibody comprises:
a heavy chain conjugated or fused to the polypeptide having at least 80% identity with the amino acid sequence that ranges from the amino acid residue (Q) at position 71 to the amino acid residue (T) at position 602 in SEQ ID NO:1 and
a light chain conjugated or fused to both the fusion protein having at least 80% of identity with amino acid sequence as set forth in SEQ ID NO:11.

16. The antibody according to claim 1, wherein the antibody is an IgG4 antibody.

17. The antibody according to claim 1, wherein the antibody is specific for CD40.

18. The antibody of claim 17 wherein:
the antibody specific for CD40 comprises:
a heavy chain comprising complementarity determining regions CDR1H having the amino acid sequence GFTFSDYYMY (SEQ ID NO:12), CDR2H having the amino acid sequence YINSGGGSTYYPDTVKG (SEQ ID NO:12), and CDR3H having the amino acid sequence RGLPFHAMDY (SEQ ID NO:14), and
a light chain comprising complementarity determining regions CDR1L having the amino acid sequence SASQGISNYLN (SEQ ID NO:15), CDR2L having the amino acid sequence YTSILHS (SEQ ID NO:16) and CDR3L having the amino acid sequence QQFNKLPPT (SEQ ID NO:17); or
the antibody specific for CD40 comprises:
a heavy chain comprising CDR1H having the amino acid sequence GYSFTGYYMH (SEQ ID NO:18), CDR2H having the amino acid sequence RINPYNGATSYNQNFKD (SEQ ID NO:19), and CDR3H having the amino acid sequence EDYVY (SEQ ID NO:20), and
a light chain comprising CDR1L having the amino acid sequence RSSQSLVHSNGNTYLH (SEQ ID NO:21), CDR2L having the amino acid sequence KVSNRFS (SEQ ID NO:22) and CDR3L having the amino acid sequence SQSTHVPWT (SEQ ID NO:23); or
the antibody specific for CD40 comprises:
a heavy chain comprising CDR1H having the amino acid sequence GYTFTDYVLH (SEQ ID NO:24), CDR2H having the amino acid sequence YINPYNDGTKYNEKFKG (SEQ ID NO:25), and CDR3H having the amino acid sequence GYPAYSGYAMDY (SEQ ID NO:26), and
a light chain comprising CDR1L having the amino acid sequence RASQDISNYLN (SEQ ID NO:27), CDR2L having the amino acid sequence YTSRLHS (SEQ ID NO:28) and CDR3L having the amino acid sequence HHGNTLPWT (SEQ ID NO:29).

19. The antibody of claim 17 wherein the antibody specific for CD40 comprises: a heavy chain with a sequence set forth as SEQ ID NO: 30 and a light chain with a sequence set forth as SEQ ID NO: 31; a heavy chain with a sequence set forth as SEQ ID NO: 32 and a light chain with a sequence set forth as SEQ ID NO: 31; a heavy chain with a sequence set forth as SEQ ID NO: 33 and a light chain with a sequence set forth as SEQ ID NO: 34; a heavy chain with a sequence set forth as SEQ ID NO: 35 and a light chain with a sequence set forth as SEQ ID NO: 36; a heavy chain with a sequence set forth as SEQ ID NO: 37 and a light chain with a sequence set forth as SEQ ID NO: 38; or a heavy chain with a sequence set forth as SEQ ID NO: 39 and a light chain with a sequence set forth as SEQ ID NO: 40.

20. The antibody of claim 1 comprising:
a heavy chain having at least 80% identity with the amino acid sequence as set forth in SEQ ID NO:47, and
a light chain having at least 80% identity with the amino acid sequence as set forth in SEQ ID NO:48 or SEQ ID NO:49.

21. (canceled)

22. A polynucleotide that encodes for the heavy chain and/or the light chain of the antibody according to claim 1; a vector comprising the polynucleotide; or a host cell transfected, infected or transformed by the polynucleotide and/or the vector.

23. (canceled)

24. (canceled)

25. A vaccine composition comprising the antibody according to claim 1.

26. A method for vaccinating a subject in need thereof against Nipah virus comprising administering to the subject a therapeutically effective amount of the antibody according to claim 1.


SEQUENCE LISTING (49 sequences)

SEQ ID NO: 1 - protein, 602 aa, Nipah henipavirus
MPAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 2 - protein, 546 aa, Nipah henipavirus
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT

SEQ ID NO: 3 - protein, 532 aa, Nipah henipavirus
MSDIFEEAASFRSYQSKLGRDGRASAATATLTTKIRIFVPATNSPELRWELTLFALDVIRSPSAAESMKVGAAFTLISMYSERPGALIRSLLNDPDIEAVIIDVGSMVNGIPVMERRGDKAQEEMEGLMRILKTARDSSKGKTPFVDSRAYGLRITDMSTLVSAVITIEAQIWILIAKAVTAPDTAEESETRRWAKYVQQKRVNPFFALTQQWLTEMRNLLSQSLSVRKFMVEILIEVKKGGSAKGRAVEIISDIGNYVEETGMAGFFATIRFGLETRYPALALNEFQSDLNTIKSLMLLYREIGPRAPYMVLLEESIQTKFAPGGYPLLWSFAMGVATTIDRSMGALNINRGYLEPMYFRLGQKSARHHAGGIDQNMANRLGLSSDQVAELAAAVQETSAGRQESNVQAREAKFAAGGVLIGGSDQDIDEGEEPIEQSGRQSVTFKREMSISSLANSVPSSSVSTSGGTRLTNSLLNLRSRLAAKAAKEAASSNATDDPAISNRTQGESEKKNNQDLKPAQNDLDFVRADV

SEQ ID NO: 4 - protein, 149 aa, synthetic construct
MQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 5 - protein, 27 aa, synthetic construct
QTPTNTISVTPTNNSTPTNNSNPKPNP

SEQ ID NO: 6 - protein, 25 aa, synthetic construct
SSVSPTTSVHPTPTSVPPTPTKSSP

SEQ ID NO: 7 - protein, 25 aa, synthetic construct
PTSTPADSSTITPTATPTATPTIKG

SEQ ID NO: 8 - protein, 25 aa, synthetic construct
TVTPTATATPSAIVTTITPTATTKP

SEQ ID NO: 9 - protein, 25 aa, synthetic construct
TNGSITVAATAPTVTPTVNATPSAA

SEQ ID NO: 10 - protein, 12 aa, synthetic construct
AEAAAKEAAAKA

SEQ ID NO: 11 - protein, 96 aa, synthetic construct
KYKIKSNPLTKDIVIKMIPNVSNMSQCTGSVMENYKTRLNGILTPIAEAAAKEAAAKAIQTKFAPGGYPLLWSFAMGVATTIDRSMGALNINRGYL

SEQ ID NO: 12 - protein, 10 aa, synthetic construct
GFTFSDYYMY

SEQ ID NO: 13 - protein, 17 aa, synthetic construct
YINSGGGSTYYPDTVKG

SEQ ID NO: 14 - protein, 10 aa, synthetic construct
RGLPFHAMDY

SEQ ID NO: 15 - protein, 11 aa, synthetic construct
SASQGISNYLN

SEQ ID NO: 16 - protein, 7 aa, synthetic construct
YTSILHS

SEQ ID NO: 17 - protein, 9 aa, synthetic construct
QQFNKLPPT

SEQ ID NO: 18 - protein, 10 aa, synthetic construct
GYSFTGYYMH

SEQ ID NO: 19 - protein, 17 aa, synthetic construct
RINPYNGATSYNQNFKD

SEQ ID NO: 20 - protein, 5 aa, synthetic construct
EDYVY

SEQ ID NO: 21 - protein, 16 aa, synthetic construct
RSSQSLVHSNGNTYLH

SEQ ID NO: 22 - protein, 7 aa, synthetic construct
KVSNRFS

SEQ ID NO: 23 - protein, 9 aa, synthetic construct
SQSTHVPWT

SEQ ID NO: 24 - protein, 10 aa, synthetic construct
GYTFTDYVLH

SEQ ID NO: 25 - protein, 17 aa, synthetic construct
YINPYNDGTKYNEKFKG

SEQ ID NO: 26 - protein, 12 aa, synthetic construct
GYPAYSGYAMDY

SEQ ID NO: 27 - protein, 11 aa, synthetic construct
RASQDISNYLN

SEQ ID NO: 28 - protein, 7 aa, synthetic construct
YTSRLHS

SEQ ID NO: 29 - protein, 9 aa, synthetic construct
HHGNTLPWT

SEQ ID NO: 30 - protein, 116 aa, synthetic construct
EVQLVQSGAEVKKPGASVKISCKASGYSFTGYYMHWVKQAHGQGLEWIGRINPYNGATSYNQNFKDRATLTVDKSTSTAYMELSSLRSEDTAVYYCAREDYVYWGQGTTVTVSSAS

SEQ ID NO: 31 - protein, 108 aa, synthetic construct
DVVMTQSPLSLPVTLGQPASISCRSSQSLVHSNGNTYLHWYQQRPGQSPRLLIYKVSNRFSGVPDRFSGSGSGTDFTLKISRVEAEDVGVYFCSQSTHVPWTFGGGTK

SEQ ID NO: 32 - protein, 116 aa, synthetic construct
EVQLVQSGAEVKKPGASVKVSCKASGYSFTGYYMHWVRQAPGQGLEWIGRINPYNGATSYNQNFKDRVTLTVDKSTSTAYMELSSLRSEDTAVYYCAREDYVYWGQGTTVTVSSAS

SEQ ID NO: 33 - protein, 123 aa, synthetic construct
EVQLQQSGPELVKPGASVKMSCKASGYTFTDYVLHWVKQKPGQGLEWIGYINPYNDGTKYNEKFKGKATLTSDKSSSTAYMELSSLTSEDSAVYYCARGYPAYSGYAMDYWGQGTSVTVSSAS

SEQ ID NO: 34 - protein, 103 aa, synthetic construct
DIQMTQTTSSLSASLGDRVTISCRASQDISNYLNWYQQKPDGTVKLLIYYTSRLHSGVPSRFSGSGSGTDYSLTISNLEQEDIATYFCHHGNTLPWTFGGGTK

SEQ ID NO: 35 - protein, 121 aa, synthetic construct
DVQLQESGPDLVKPSQSLSLTCTVTGYSITSDYSWHWIRQFPGNKLEWMGYIYYSGSTNYNPSLKSRISITRDTSKNQFFLQLNSVTTEDSATYFCARFYYGYSFFDYWGQGTTLTVSSAS

SEQ ID NO: 36 - protein, 102 aa, synthetic construct
QIVLTQSPAFMSASPGEKVTMTCSASSSVSYMHWYQQKSGTSPKRWIYDTSKLASGVPARFSGSGSGTSYSLTISSMEAEDAATYYCQQWSSNPLTFGAGTK

SEQ ID NO: 37 - protein, 128 aa, synthetic construct
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSAS

SEQ ID NO: 38 - protein, 103 aa, synthetic construct
DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTK

SEQ ID NO: 39 - protein, 121 aa, synthetic construct
EVQLVESGGGLVQPGGSLKLSCATSGFTFSDYYMYWVRQAPGKGLEWVAYINSGGGSTYYPDTVKGRFTISRDNAKNTLYLQMNSLRAEDTAVYYCARRGLPFHAMDYWGQGTLVTVSSAS

SEQ ID NO: 40 - protein, 103 aa, synthetic construct
DIQMTQSPSSLSASVGDRVTITCSASQGISNYLNWYQQKPGKAVKLLIYYTSILHSGVPSRFSGSGSGTDYTLTISSLQPEDFATYYCQQFNKLPPTFGGGTK

SEQ ID NO: 41 - protein, 81 aa, synthetic construct
SVKMSCKASGYTFTDYVISWVKQRTGQGLEWIGDIYPGSGYSFYNENFKGKATLTADKSSTTAYMQLSSLTSEDSAVYFCA

SEQ ID NO: 42 - protein, 76 aa, synthetic construct
ASISCRSSQSLVHSNGNTYLHWYLQKPGQSPKLLIYKVSNRFSGVPDRFSGSGSGTNFTLKISRVEAEDLGLYFCS

SEQ ID NO: 43 - protein, 82 aa, synthetic construct
SSVKMSCKASGYTFTDYVISWVKQRTGQGLEWIGDIYPGSGYSFYNENFKGKATLTADKSSTTAYMQLSSLTSEDSAVYFCA

SEQ ID NO: 44 - protein, 74 aa, synthetic construct
VTLTCRSSTGAVTTSNYANWVQEKPDHLFTGLIGGTNNRVSGVPARFSGSLIGDKAALTITGAQTEDEAIYFCA

SEQ ID NO: 45 - protein, 456 aa, synthetic construct
QVQLQQSGAELVRPGASVTLSCKASGYTFIDHDMHWVQQTPVYGLEWIGAIDPETGDTGYNQKFKGKAILTADKSSRTAYMELRSLTSEDSAVYYCTIPFYYSNYSPFAYWGQGALVTVSAAKTTAPSVYPLAPVCGGTTGSSVTLGCLVKGYFPEPVTLTWNSGSLSSGVHTFPALLQSGLYTLSSSVTVTSNTWPSQTITCNVAHPASSTKVDKKIEPRVPITQNPCPPLKECPPCADLLGGPSVFIFPPKIKDVLMISLSPMVTCVVVDVSEDDPDAQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGKAS

SEQ ID NO: 46 - protein, 213 aa, synthetic construct
QIVLSQSPAILSASPGEKVTMTCRASSSVSYMHWYQRKPGSSPKPWIYATSNLASGVPARFSGSGSGTSYSLTISRVEAEDAATYYCQQWSSNPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 47 - protein, 1030 aa, synthetic construct
MGWSLILLFLVAVATRVHSEVQLVESGGGLVQPGGSLKLSCATSGFTFSDYYMYWVRQAPGKGLEWVAYINSGGGSTYYPDTVKGRFTISRDNAKNTLYLQMNSLRAEDTAVYYCARRGLPFHAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKASQTPTNTISVTPTNNSTPTNNSNPKPNPASQNYTRSTDNQAMIKDALQSIQQQIKGLADKIGTEIGPKVSLIDTSSTITIPANIGLLGSKISQSTASINENVNEKCKFTLPPLKIHECNISCPNPLPFREYKPQTEGVSNLVGLPNNICLQKTSNQILKPKLISYTLPVVGQSGTCITDPLLAMDEGYFAYSHLEKIGSCSRGVSKQRIIGVGEVLDRGDEVPSLFMTNVWTPSNPNTVYHCSAVYNNEFYYVLCAVSVVGDPILNSTYWSGSLMMTRLAVKPKNNGESYNQHQFALRNIEKGKYDKVMPYGPSGIKQGDTLYFPAVGFLVRTEFKYNDSNCPIAECQYSKPENCRLSMGIRPNSHYILRSGLLKYNLSDEENSKIVFIEISDQRLSIGSPSKIYDSLGQPVFYQASFSWDTMIKFGDVQTVNPLVVNWRDNTVISRPGQSQCPRFNKCPEVCWEGVYNDAFLIDRINWISAGVFLDSNQTAENPVFTVFKDNEVLYRAQLASEDTNAQKTITNCFLLKNKIWCISLVEIYDTGDNVIRPKLFAVKIPEQCTLK

SEQ ID NO: 48 - protein, 214 aa, synthetic construct
DIQMTQSPSSLSASVGDRVTITCSASQGISNYLNWYQQKPGKAVKLLIYYTSILHSGVPSRFSGSGSGTDYTLTISSLQPEDFATYYCQQFNKLPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 312 aa, synthetic construct
DIQMTQSPSSLSASVGDRVTITCSASQGISNYLNWYQQKPGKAVKLLIYYTSILHSGVPSRFSGSGSGTDYTLTISSLQPEDFATYYCQQFNKLPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECASKYKIKSNPLTKDIVIKMIPNVSNMSQCTGSVMENYKTRLNGILTPIAEAAAKEAAAKAIQTKFAPGGYPLLWSFAMGVATTIDRSMGALNINRGYL